(19) United States
(12) Patent Application Publication
(10) Pub. No.: US 2005/0069572 A1
(43) Pub. Date: Mar. 31, 2005

(54) Multi-layered polymerizing hydrogels for tissue regeneration

(75) Inventors: Christopher G. Williams, Baltimore, MD (US); Blanka Sharma, Baltimore, MD (US); Jennifer H. Elisseeff, Baltimore, MD (US)

Correspondence Address: GRIFFIN & SZIPL, PC, SUITE PH-1, 2300 NINTH STREET, SOUTH, ARLINGTON, VA, 22204, US

(73) Assignee: Jennifer H. Elisseeff, Baltimore, MD (US)

(21) Appl. No.: 10/681,753

(22) Filed: Oct. 9, 2003

Related U.S. Application Data
(60) Provisional application No. 60/416,882, filed on Oct. 9, 2002.
(60) Provisional application No. 60/416,881, filed on Oct. 9, 2002.

(51) Int. Cl. A61K 45/00

(52) U.S. Cl. 424/426; 424/93.7

(57) ABSTRACT

A multi-layered tissue construct includes: a first layer comprising a first hydrogel; and a second layer comprising a second hydrogel, wherein the first layer is connected to the second layer at a first transition zone and wherein at least one of the first layer and the second layer further comprises a component selected from the group consisting of cells and a bioactive substance. Another multi-layered tissue construct includes: a first layer comprising a first hydrogel; a second layer comprising cells of a first type, wherein the second layer is disposed on the first layer; and a third layer comprising a second hydrogel and optionally cells of the first type encapsulated in the second hydrogel, wherein the third layer is disposed on the second layer. Methods for producing these multi-layered tissue constructs are also disclosed.

### CROSS-REFERENCE TO RELATED APPLICATION

This application claims benefit under 35 U.S.C. 119(e) of U.S. Provisional Applications 60/416,882 and 60/416,881, both filed Oct. 9, 2002, the entire disclosure of which is hereby incorporated by reference

This application is related to a utility patent application claiming priority to U.S. Provisional Application No. 60/413,152 (filed Sep. 25, 2002), entitled “Cross-linked polymer matrices, and methods of making and using same,” and filed on Sep. 25, 2003, the entire disclosure of which is hereby incorporated by reference.

This application is also related to a utility patent application claiming priority to U.S. Provisional Application No. 60/416,881 (filed Oct. 9, 2002), entitled “Tissue-initiated photopolymerization fro enhanced tissue-biomaterial integration,” and filed on Oct. 9, 2003, the entire disclosure of which is hereby incorporated by reference.

### FIELD OF THE INVENTION

The present invention pertains broadly to a method of tissue engineering. More specifically, the present invention pertains to a method of producing a multi-layered tissue construct for use as tissue engineering scaffolds with integrated, separate, layers of hydrogel. The invention further relates to a multiple layer construct produced according to the method, particularly one comprising one or more different cell types in the construct. The invention also relates to a method for replacing lost or damaged tissue in a host recipient or patient using the multiplayer construct of the present invention.

### BACKGROUND OF THE INVENTION

Bioengineered tissues offer a solution for the restoration of damaged organs and tissues in recipient hosts and patients, especially considering the limited availability of human donor tissue. In particular, there is a large demand for structural tissues such as cartilage and bone. These tissues have complex architectures, and it is advantageous to closely mimic these structures in order to obtain a structurally and functionally equivalent tissue substitute. In other words, when bioengineering substitute tissues, it would be advantageous to reproduce, as closely as possible, the natural cellular architecture of the tissue being replaced.

Fabricating polymers in vitro or in vivo provides many advantages for a variety of biomedical applications, such as tissue engineering. The first biomedical applications of photopolymerizable materials occurred in the dental field, where such materials were used as sealants on teeth and for dental restoration. Photopolymerization of photopolymerizable mixtures can be used to synthesize hydrogels, which are crosslinked hydrophilic polymer networks capable of holding a large volume fraction of water. This high water content enables efficient transport of nutrients and waste products, which makes these hydrogels attractive as matrices for supporting living cells when creating tissue scaffolds.

In the field of tissue engineering, polymerizing hydrogels additionally provide attractive scaffolds because of their biocompatibility and ability to be subsequently administered in vivo in a minimally invasive manner as discussed in U.S. Pat. No. 5,399,665 to Barrera et al. Hydrogels can be polymerized using light, UV radiation, a redox agent (e.g. sodium thiosulfate in combination with sodium persulfate), or by using some other suitable polymerization initiator such as a divalent cation like calcium. Photopolymerizing hydrogels are currently being studied for use in minimally invasive surgical procedures, including the prevention of postsurgical tissue adhesions and restenosis after angioplasty, because the polymerization initiator, either light or UV radiation, can be conveniently administered through a surgical scope. Furthermore, there have been recent innovations involving photopolymerizable hydrogels in the fields of drug delivery and tissue engineering as taught by Hubbell et al. in U.S. Pat. No. 5,567,435

Previous studies using photopolymerizing poly (ethylene oxide) dimethacrylate based hydrogels have demonstrated the ability of these gels to encapsulate chondrocytes, which eventually produced cartilaginous tissue. For example, see Elisseeffet al., Proc. Natl. Acad. Sci. USA, vol. 96, pp. 3104-3107, 1999, herein incorporated in is entirety by reference.

A drawback to conventional cell encapsulation strategies, however, is that the cells are homogenously encapsulated throughout the hydrogel. This homogenous structure does not accurately reproduce the physiologic cellular organization of natural tissues, which generally consists of a highly organized arrangement of different cell types in an extracellular matrix. In other words, natural tissues generally do not consist of a single cell type homogenously dispersed in an extracellular matrix.

Cartilage is one example of a naturally occurring tissue type that has various layers and which is not entirely satisfactorily approximated by a non-layered tissue construct. Specifically, as shown in FIG. 8, naturally occurring mammalian cartilage C includes chondrocytes ch encapsulated by an extracellular matrix M. Cartilage C is organized into three different layered zones, which are the superficial STZ zone 1, the middle zone 2, and the deep zone 3. Roughly, when considering the thickness of hyaline cartilage at a diaphysial joint, the superficial STZ zone 1 makes up about 10-20% of the thickness of the cartilage C, whereas the middle zone 2 and the deep zone 3 make up about 40-60% and 30%, respectively, of the thickness of the cartilage between the articular surface 7 and the tide mark 6. Below the tide mark 6, there is a zone of calcifying cartilage known as the calcified zone 4 under which is subchondral bone 5.

The phenotype of chondrocyte cells in each zone 1, 2, 3, and the biochemical milieu of each zone, is different and provides a unique architecture leading to the great mechanical strength of cartilage. For example, the chondrocytes in zone 1 are densely packed and there is less extracellular matrix M, which provides a relatively weak but fluid impermeable zone that regulates fluid and proteoglycan flow through the tissue and that is directly related to mechanical function. On the other hand, the chondrocytes in the deep zone 3 are larger and produce more matrix M than zone 1 chondrocytes, which gives cartilage C its compressive strength. It was recently discovered by the present inventors that the superficial chondrocytes in zone 1 interact with the deep chondrocytes in zone 3 to slow the rate of proliferation of the deep chondrocytes and to cause them to produce more matrix M (unpublished data).

Hyaline cartilage C is typically found on the ends of bone at diarthrodial joints and serves to coat the surface of the bone ends to lessen friction and provide a shock absorber. However, as individuals age, the relatively weak superficial STZ zone 1 is damaged or erodes and the process of osteoarthritis begins. As this process progresses, the middle zone 2 and the deep zone 3 can be damaged or eroded even to the point of exposing subchondral bone 5. Because there are many patients with osteochondral lesions where both cartilage and bone must be replaced, there is a need for a multi-layered tissue construct, usable as a tissue substitute, that more closely mimics the architecture of cartilage than conventional non-layered tissue constructs.

It is known that mixed cell populations augment the function of the various cell types through the use of chemical messengers and biological signals that affect neighboring cell function. Consequently, conventional homogenously dispersed, non-layered, single cell type tissue constructs known in the prior art cannot recreate the augmentation of cellular function that occurs naturally in heterogeneous cellular communities within the physiologic architecture of naturally occurring mammalian tissue. Some tissue constructs, such as the tissue construct 10 taught by Elisseeff et al., Proc. Natl. Acad. Sci. USA, vol. 96, pp. 3104-3107, 1999, or the tissue construct taught by Griffith-Cima et al. in U.S. Pat. No. 5,709,854, embedded chondrocytic cells from all cartilage zones 1, 2 and 3 in a hydrophilic hydrogel 15. However, such constructs homogenously distribute superficial zone chondrocytes 11 with both middle zone chondrocytes 12 and deep zone chondrocytes 13 in a non-layered fashion as shown in FIG. 9. In this respect, the prior Elisseeff et al. tissue construct incorporated multiple cell types in a hydrogel polymerized using photopolymerization, but it did not attempt to mimic the layered architecture of natural cartilage.

Other examples of prior non-layered tissue constructs are also known. For example, Vacanti et al. (U.S. Pat. No. 6,123,727) teach using tenocytes or chondrocytes encapsulated in a biodegradable polymer to create an engineered tendon or ligament.

Thus, conventional non-layered tissue constructs do not closely mimic the cellular architecture of naturally occurring tissues, which may limit the usefulness of these tissue substitutes. On the other hand, it is an object of the present invention to take advantage of the ability to temporally and spatially control the polymerization reaction of polymerizable material to make hydrogels with multiple layers containing one or more different cell types. In this way, multi-layered tissue constructs that more closely resemble the actual cellular organization of the target tissue, such as cartilage or bone, can be manufactured either in vitro or in vivo.

The present invention endeavors to provide multi-layered tissue constructs, using polymerizable hydrogels, engineered to contain multiple layers of different cell types in order to more closely mimic the complex tissue architecture of physiological tissues. Thus, the present invention provides a multi-layered tissue construct, which more closely resembles the complex cellular architecture of physiologic tissues than non-layered tissue constructs, and a method for making these multi-layered tissue constructs.

Accordingly, it is an object of the present invention to overcome the disadvantages of prior non-layered tissue constructs while maintaining the advantages of the prior non-layered tissue constructs, and even improving thereon.

Another object of the present invention is to provide multi-layered tissue constructs that are biocompatible with living tissues.

Another object of the present invention is to provide a multi-layered tissue construct that more closely resembles the structure of physiologically layered tissues than the non-layered tissue constructs of the prior art.

Another object of the present invention is to provide a multi-layered tissue construct, wherein each layer includes cells predominately of a certain cell type so as to more closely resemble physiologically layered tissues than the non-layered tissue constructs of the prior art.

Another object of the present invention is to provide multi-layered tissue constructs usable as tissue engineering scaffolds, wherein the layers are integrated, but separate, and each layer includes predominately a single cell type embedded in a hydrogel.

Another object of the present invention is to provide a multi-layered tissue construct that includes separate layers for predominately superficial, middle and deep zone chondrocytes so as to more closely resemble natural cartilage and osteochondral composite tissues consisting of bone and cartilage.

Another object of the present invention is to provide a method of making or creating a multi-layered tissue construct that utilizes a photopolymerizing hydrogel so the method can be performed by injecting a photopolymer-cell suspension into a mammalian joint in a minimally invasive fashion (i.e., during arthroscopic joint surgery) so the multi-layered construct is synthesized in situ.

Another object of the present invention is to provide a method of making or creating a multi-layered tissue construct that can be applied to the in situ formation of a tissue scaffold in the joint environment of a mammal using arthroscopic implantation techniques.

Another object of the present invention is to provide an engineered multi-layered tissue construct that can incorporate a bone layer to help anchor tissue implants an improve integration of implants with host tissues.

### SUMMARY OF THE INVENTION

In accordance with the above objectives, the present invention provides, in a first method embodiment, a method of producing a multi-layered tissue construct is claimed that includes the steps of: (a) providing a first polymerizable mixture, including, optionally, a first polymerization initiator; (b) providing a second polymerizable mixture, including, optionally, a second polymerization initiator; (c) wherein one of the first and second mixtures comprises a component selected from the group consisting of cells and a bioactive substance; (d) placing a volume of the first mixture in a space, then crosslinking the first mixture for a first predetermined time until the first mixture forms an at least partially gelled first layer; and (e) placing a volume of the second mixture in the space with the at least partially gelled first layer, then crosslinking the second mixture for a second predetermined time until the second mixture is at least partially gelled to form a second layer.

In accordance with a second method embodiment of the present invention, the first method embodiment is modified so that one of the first and second mixtures comprises cells.

In accordance with a third method embodiment of the present invention, the first method embodiment is modified to further include the step of adding a suspension of cells to a surface of the at least partially gelled first layer, before the step of placing the volume of the second mixture in the space.

In accordance with a fourth method embodiment of the present invention, the first method embodiment is modified so that the bioactive substance is selected from the group consisting of: a nutrient, a cellular mediator, a growth factor, a compound which induces cellular differentiation, a bioactive polymer, a gene vector, or a pharmaceutical.

In accordance with a fifth method embodiment of the present invention, the first method embodiment is modified so the step of providing the first mixture includes mixing the first polymerizable mixture with first cells to form a first polymer-cell suspension, and the step of providing the second mixture includes mixing the second polymerizable mixture with second cells to form a second polymer-cell suspension.

In accordance with a sixth method embodiment of the present invention, the fifth method embodiment further includes the step of additionally crosslinking the first mixture and the second mixture until the first layer and the second layer further polymerize to form an integrated multi-layered gel.

In accordance with a seventh method embodiment of the present invention, the fifth method embodiment is modified so the first cells and the second cells are selected from the group of cell types consisting of superficial zone chondrocytes, middle zone chondrocytes, and deep zone chondrocytes.

In accordance with a eighth method embodiment of the present invention, the seventh method embodiment is modified so the first cells are a cell type different from the second cells.

In accordance with a ninth method embodiment of the present invention, the eighth method embodiment is modified so the first cells are deep zone chondrocytes and the second cells are superficial zone chondrocytes.

In accordance with a tenth method embodiment of the present invention, the seventh method embodiment is modified to further include the steps of: harvesting mammalian articular cartilage and excising tissue specimens corresponding to an upper zone, a middle zone and a deep zone of the cartilage; and separately digesting the tissue specimens from the upper zone, the middle zone and the deep zone respectively to isolate upper zone chondrocytes, middle zone chondrocytes and deep zone chondrocytes.

In accordance with a eleventh method embodiment of the present invention, the fifth method embodiment is modified so that the cell concentration of each suspension is approximately 20 million cells/cc.

In accordance with a twelfth method embodiment of the present invention, the sixth method embodiment is modified to further include the step of: incubating the multi-layered gel in a complete media for a predetermined incubation period to form the multi-layered tissue construct.

In accordance with a thirteenth method embodiment of the present invention, the fifth method embodiment is modified to further include the steps of: providing a third polymerizable mixture, including, optionally, a third polymerization initiator, wherein the third polymerizable mixture is mixed with third cells to prepare a third polymer-cell suspension; and placing a volume of the third mixture in the space with the at least partially gelled first layer and the at least partially gelled second layer, then crosslinking the third mixture for a third predetermined time until the third mixture is at least partially gelled to form a third layer.

In accordance with a fourteenth method embodiment of the present invention, the thirteenth method embodiment is modified to further include the step of: additionally crosslinking the first layer, the second layer and the third layer to form an integrated multi-layered gel.

In accordance with a fifteenth method embodiment of the present invention, the fourteenth method embodiment is modified so the first cells, the second cells and the third cells are selected from the group of cell types consisting of superficial zone chondrocytes, middle zone chondrocytes, and deep zone chondrocytes.

In accordance with a sixteenth method embodiment of the present invention, the fifteenth method embodiment is modified to so the first cells, the second cells and the third cells are selected to be different cell types.

In accordance with a seventeenth method embodiment of the present invention, the fifteenth method embodiment is modified so the first cells are deep zone chondrocytes, the second cells are middle zone chondrocytes, and the third cells are superficial zone chondrocytes.

In accordance with an eighteenth method embodiment of the present invention, the seventeenth method embodiment is modified to further include the step of: incubating the multi-layered gel in a complete media for a predetermined incubation period to form the multi-layered tissue construct.

In accordance with a nineteenth method embodiment of the present invention, the thirteenth method embodiment is modified to further include the steps of additionally crosslinking the first layer, the second layer and the third layer until the first layer, the second layer and the third layer completely polymerize to form a multi-layered gel; and optionally incubating the multi-layered gel in a complete media for a predetermined period of time to form the multi-layered tissue construct.

In accordance with a twentieth method embodiment of the present invention, the fifth method embodiment is modified to so the first polymerizable mixture and the second polymerizable mixture both include photopolymerizable poly~ethylene glycol) diacrylate dissolved in solvent, which is phosphate buffered saline, to make a 10% w/v solution, and the first polymerization initiator is added to the first mixture and the second polymerization initiator is added to the second mixture, wherein both the first polymerization initiator and the second polymerization initiator are the same photoinitiator, and each suspension has a concentration of 20 million cells/cc.

In accordance with a twenty-first method embodiment of the present invention, the twentieth method embodiment is modified so the photoinitiator is Igracure 2959 mixed to a concentration of 0.05% w/v in each suspension.

In accordance with a twenty-second method embodiment of the present invention, the twenty-first method embodiment is modified so crosslinking of the first polymerizable mixture is controlled by exposure to external radiation and crosslinking of the second polymerizable mixture is controlled by exposure to the external radiation.

In accordance with a twenty-third method embodiment of the present invention, the fifth method embodiment is modified so the third cells are also mixed in the first polymerizable mixture with the first cells when forming the first polymer-cell suspension.

In accordance with a first apparatus embodiment of the present invention, a multi-layered tissue construct is claimed that includes: (a) a first layer comprising a first hydrogel; and (b) a second layer comprising a second hydrogel, wherein the first layer is connected to the second layer at a first transition zone and wherein at least one of the first layer and the second layer further comprises a component selected from the group consisting of cells and a bioactive substance.

In accordance with a second apparatus embodiment of the present invention, the first apparatus embodiment is modified so the first layer comprises cells of a first cellular type encapsulated in the first hydrogel.

In accordance with a third apparatus embodiment of the present invention, the second apparatus embodiment is modified so the second layer comprises cells of a second cellular type encapsulated in the second hydrogel, and the first cell type is different from the second cell type.

In accordance with a fourth apparatus embodiment of the present invention, the third apparatus embodiment is modified to include a third layer comprising cells of a third cellular type encapsulated in a third hydrogel, wherein a second transition zone connects the third layer to the second layer, and the third cell type is different from the second cell type.

In accordance with a fifth apparatus embodiment of the present invention, the fourth apparatus embodiment is modified so the first cell type is a deep zone chondrocyte, the second cell type is a middle zone chondrocyte, and the third cell type is a superficial zone chondrocyte.

In accordance with a sixth apparatus embodiment of the present invention, the fifth apparatus embodiment is modified so the first hydrogel, the second hydrogel and the third hydrogel include photopolymerized poly(ethylene glycol) diacrylate.

In accordance with a seventh apparatus embodiment of the present invention, the third apparatus embodiment is modified so the first cell type is a deep zone chondrocyte and the second cell type is a superficial zone chondrocyte.

In accordance with an eighth apparatus embodiment of the present invention, the seventh apparatus embodiment is modified so the first hydrogel and the second hydrogel both include photopolymerized poly(ethylene glycol) diacrylate.

In accordance with a ninth apparatus embodiment of the present invention, the third apparatus embodiment is modified so the first cell type is a stem cell and the second cell type is an educator cell.

In accordance with a tenth apparatus embodiment of the present invention, the ninth apparatus embodiment is modified so the first hydrogel and the second hydrogel both include photopolymerized poly(ethylene glycol) diacrylate.

In accordance with an eleventh apparatus embodiment of the present invention, the ninth apparatus embodiment is modified so the educator cell is a chondrocyte and the stem cell is either an embryonic stem cell or a mesenchymal stem cell harvested from bone marrow.

In accordance with a twelfth apparatus embodiment of the present invention, the second apparatus embodiment is modified so the first layer further comprises cells of a second cellular type encapsulated in the first hydrogel.

In accordance with a thirteenth apparatus embodiment of the present invention, the first apparatus embodiment is modified so the first layer comprises a bioactive substance selected from the group consisting of: a nutrient, a cellular mediator, a growth factor, a compound which induces cellular differentiation, a bioactive polymer, a gene vector, or a pharmaceutical.

In accordance with a fourteenth apparatus embodiment of the present invention, the second apparatus embodiment is modified so the first layer also includes a bioactive substance.

In accordance with a fifteenth apparatus embodiment of the present invention, the second apparatus embodiment is modified so the second layer includes a bioactive substance.

In accordance with a sixteenth apparatus embodiment of the present invention, a multi-layered tissue construct is claimed that includes: (a) a first layer comprising a first hydrogel; (b) a second layer comprising cells of a first type, wherein the second layer is disposed on the first layer; and (c) a third layer comprising a second hydrogel and optionally cells of the first type encapsulated in the second hydrogel, wherein the third layer is disposed on the second layer.

In accordance with a seventeenth apparatus embodiment of the present invention, the sixteenth apparatus embodiment is modified so the second layer is connected to the first layer through an abrupt transition zone and the second layer is connected to the third layer through a smooth transition zone.

In accordance with an eighteenth apparatus embodiment of the present invention, the seventeenth apparatus embodiment is modified so the cells of the first type are disposed predominantly between the abrupt transition zone and the smooth transition zone.

In accordance with a nineteenth apparatus embodiment of the present invention, the sixteenth apparatus embodiment is modified so the third layer includes cells of the first type dispersed throughout the third layer.

In accordance with a twentieth apparatus embodiment of the present invention, the sixteenth apparatus embodiment is modified so cells of the first type are selected from the group consisting of: embryonic stem cells and mesenchymal stem cells.

In accordance with a twenty-first apparatus embodiment of the present invention, the sixteenth apparatus embodiment is modified so the first hydrogel and the second hydrogel are made of the same material.

In accordance with a twenty-second apparatus embodiment of the present invention, the twenty-first apparatus embodiment is modified so the material is formed by the photopolymerization of a polymer selected from the group consisting of: poly(ethylene glycol) diacrylate and poly(ethylene oxide) diacrylate.

In accordance with a twenty-third apparatus embodiment of the present invention, the sixteenth apparatus embodiment is modified so one or more of the first layer and the second layer further comprises a bioactive substance.

In accordance with a twenty-fourth apparatus embodiment of the present invention, a multi-layered tissue construct is claimed that is made by the process including the steps of: (a) placing a first polymerizable mixture in a space and crosslinking the first polymerizable mixture to produce an at least partially gelled first hydrogel layer; (b) placing a cell suspension on the first hydrogel layer, wherein the cell suspension includes cells of a first type to form a cell layer; (c) placing a volume of a second polymerizable mixture on the cell layer; and (d) crosslinking the second polymerizable mixture to produce an at least partially gelled second hydrogel layer integrated with the cell layer and the first hydrogel layer.

In accordance with a twenty-fifth apparatus embodiment of the present invention, the twenty-fourth apparatus embodiment is modified so the first polymerizable mixture and the second polymerizable mixture comprise the same polymer selected from the group consisting of: poly(ethylene glycol) diacrylate and poly(ethylene oxide) diacrylate.

In accordance with a twenty-sixth apparatus embodiment of the present invention, the twenty-fifth apparatus embodiment is modified so a photoinitiator is dissolved in the first polymerizable mixture and a photoinitiator is dissolved in the second polymerizable mixture so that the first polymerizable mixture is crosslinked when exposed to external radiation and the second polymerizable mixture is crosslinked when exposed to external radiation.

In accordance with a twenty-seventh apparatus embodiment of the present invention, the twenty-sixth apparatus embodiment is modified so the cells of a first type are adult stem cells.

In accordance with a twenty-eighth apparatus embodiment of the present invention, the twenty-fourth apparatus embodiment is modified so cells of the first type are suspended in the second polymerizable mixture.

In accordance with a twenty-ninth apparatus embodiment of the present invention, the twenty-fourth apparatus embodiment is modified so the cells of a second type are suspended in the second polymerizable mixture.

Further objects, features and advantages of the present invention will become apparent from the Detailed Description of the Illustrative Embodiments, which follows, when considered together with the attached drawings.

### BRIEF DESCRIPTION OF THE DRAWINGS

FIG. 1 schematically illustrates a multi-layered tissue construct having two layers in accordance with one embodiment of the present invention.

FIG. 2 schematically illustrates a multi-layered tissue construct having three layers, including a dense cell layer, in accordance with another embodiment of the present invention.

FIG. 3 schematically illustrates a magnified view of the transition zone in region A of FIG. 2.

FIG. 4 schematically illustrates a multi-layered tissue construct having three layers in accordance with another embodiment of the present invention.

FIG. 5 schematically illustrates a multi-layered tissue construct having three layers, including one dense cell layer sandwiched between two hydrogel layers, in accordance with another embodiment of the present invention.

FIG. 6 is an outline of the steps of the general method for making a multi-layered tissue construct in accordance with the present invention.

FIG. 7 is a picture representation of the steps in accordance with the method for making a multi-layered tissue construct in accordance with the present invention, wherein the hydrogels are formed by crosslinking photopolymerizable mixtures when exposed to external radiation.

FIG. 8 is a schematic illustration of the zones in articular cartilage (prior art).

FIG. 9 is a schematic of a non-layered, homogenous prior art tissue construct.

FIG. 10 is a picture of a magnified view of region B in FIG. 5.

FIG. 11 provides growth curves of the cells from different cartilage zones and the summary of the growth kinetic study. (A) Growth curves of primarily isolated chondrocytes. (B) Growth curves of passaged cells (passage, PO). (C) Initial population doublings defined as the number of population doubling for the first 3 days after plating. (D) Population doubling time (*p<0.05 and **p<0.01).

FIG. 12 corresponds to the RT-PCR of cartilage specific markers, wherein β-Actin and GAPDH were displayed as the internal control (U=upper chondrocytes, M=middle chondrocytes, L=lower chondrocytes).

### DETAILED DESCRIPTION OF THE ILLUSTRATIVE EMBODIMENTS

The multi-layer tissue construct of the present invention, and the method for making this construct, involve at least a two-layered structure. A multi-layer tissue construct involving three or more hydrogel layers also falls within the scope of the present invention. To facilitate an easy understanding of the invention, the method embodiments will be described first, then the product of the method is described, which is a multi-layer tissue construct usable as a tissue implant or as a tissue scaffold.

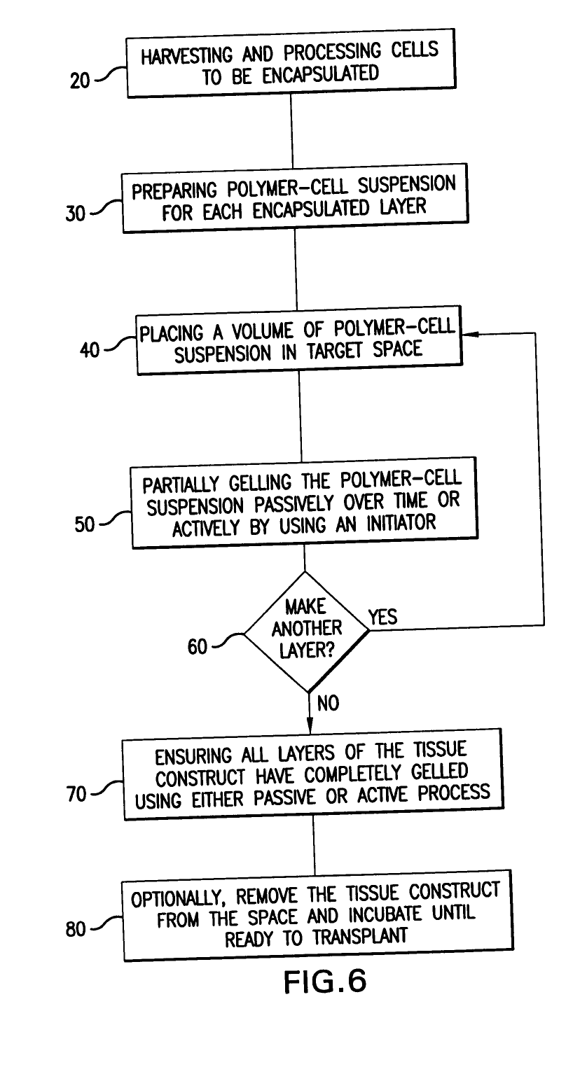

The steps in the method, in accordance with the present invention, for engineering a multi-layered tissue construct are outlined in FIG. 6. The method is briefly summarized as follows. First, cells corresponding to the cell types of the layered target tissue are harvested. Second, a polymer-cell suspension is prepared for each layer having a specific and different cell type. Third, in a sequential manner, a predetermined volume of each polymer-cell suspension is placed in a “space” (i.e., a cavity in a mold or a cavity in tissue) and partially gelled (with or without use of a polymerization initiator) before adding the next layer. Once all of the layers have been placed in the space and partially gelled, all of the partially gelled layers are allowed to undergo additional crosslinking until all of the layers have further or completely gelled. Lastly, the multi-layered tissue construct can be further incubated to prepare the multi-layer tissue construct for transplant when created in vitro.

Definitions

For the purposes of this disclosure, the following terms are defined.

A multi-layered tissue construct is defined broadly as either a multi-layered construct mimicking the structure of a multi-layered tissue or as a multi-layered construct that promotes the regeneration of tissue. A multi-layered tissue construct in accordance with this definition may, or may not, include live cells.

A polymerizable mixture as used herein is any suitable polymerizable polymer, monomer, or mixture of monomers and polymers that forms: a covalently crosslinked network, with or without the presence of a polymerization initiator, an ionically crosslinked network, or blends of covalently and ionically crosslinked networks. Polymerizable mixtures in accordance with the present invention must be able to form polymerized networks that are non-toxic to the cells being encapsulated.

A photopolymerizable polymer is any suitable polymer that forms a covalently crosslinked network using radiation provided by an external source, or blends of covalently and ionically crosslinkable or hydrophilic polymers which, when exposed to radiation from an external source, form semi-interpenetrating networks having cells suspended therein. Photopolymerizable mixtures in accordance with the present invention must be able to form polymerized networks that are non-toxic to the cells being encapsulated.

A polymerization initiator is any substance that initiates crosslinking of the polymer to form a hydrogel network, and includes redox agents, divalent cations such as calcium, and substances that form active species when exposed to visible light and/or UV radiation. A photoinitiator is a specific type of polymerization initiator that generates an active species when exposed to UV light and/or visible light, and can be used to initiate polymerization (i.e., crosslinking) of the photopolymerizable mixtures. Polymerization initiators and photoinitiators in accordance with the present invention must be non-toxic to the cells being encapsulated when used in the amounts required to initiate crosslinking of the polymerizable mixtures.

A hydrogel for encapsulating living cells is a hydrophilic polymer network with a high water content. Such hydrogels in accordance with the present invention, may have, for example, a water content greater than about 70-90%. Such hydrogels in accordance with the present invention are non-toxic to the encapsulated cells and permit the movement of nutrients to the cells, and waste products away from the cells, through the polymer network. It is noted that the multi-layered tissue constructs in accordance with the present invention can include one or more layers made with a hydrogel layer having a water content less than 70%, but such low water content hydrogels are used to provide barrier layers or support layers and are not used to encapsulate living cells.

The term “space,” as used to described the location of where hydrogels are formed, is defined broadly and may include a cavity formed in a mold, a cavity surgically formed in tissue, or a naturally existing cavity in tissue that can be surgically accessed (i.e., a joint space or joint defect).

Source of Cells

The first step 20 of the method for making, or creating, a multi-layer tissue construct in accordance with the present invention involves obtaining specific cell types to be encapsulated by the hydrogel. Generally, specific cell types of interest are harvested directly from a donor, or are harvested from cell culture of cells from a donor, or are harvested from established cell culture lines that originated from a donor. In the most preferred embodiments, autologous cells are used. However, the scope of the present invention includes the use of cells from the same mammalian species, and preferably having the same immunologic profile. When the target host is a human patient, preferably the cells will be harvested from the patient or a close relative, although cells donated by cadavers may also be suitable.

While the present invention will be described below in terms of a particular illustrative embodiment (i.e., a multi-layered tissue construct utilizing chondrocytes, stem cells, etc.), the present invention is not limited to any specific cell types. The present invention can be used to implant many different types of organ cells to include chondrocytes, osteoblasts, other cells that form bone, muscle cells, fibroblasts, hepatocytes, islet cells, cells of intestinal origin, cells of kidney origin, stem cells, and other cells acting primarily to synthesize and secrete, or to metabolize materials as described in U.S. Pat. No. 6,224,893 B1 to Langer et al., the entire disclosure of which is incorporated herein by reference.

Preparation of Polymer-Cell Suspensions

The second step 30 in the method for making, or creating, a multi-layer tissue construct in accordance with the present invention involves preparing polymer-cell suspensions for each layer of the multi-layered tissue construct. In certain embodiments of the multi-layer tissue construct in accordance with the present invention there can be at least one hydrogel layer that includes the hydrogel formed by polymerization of the polymerizable polymer but which does not include cells. In certain other embodiments of the multi-layer tissue construct in accordance with the present invention, there can be at least one cell layer that includes cells of a specific type that were not suspended in the polymer. To facilitate an understanding of the basic method in accordance with the present invention, the method outlined in FIG. 6 will be described first and modifications will be subsequently described.

The hydrogel solution is prepared, for example, by mixing 10% weight/volume (w/v) of the polymerizable polymer in sterile phosphate buffered saline (PBS), which is a suitable solvent, adjusted to a pH of about 7.4. Preferably, the polymer is either photopolymerizable poly(ethylene glycol) diacrylate (PEGDA) or photopolymerizable poly(ethylene oxide) diacrylate (PEODA), which are commercially available from Shearwater Corporation, Huntsville, Ala.).

Optionally, various additives can be included in the hydrogel solution such as 100 U/ml of penicillin and 100 μg/ml streptomycin to inhibit microbacterial contamination. However, these are not the only bioactive additives that can be included in the hydrogel solution. For example, the bioactive additives could include, singly or in combination, growth factors, cell differentiation factors, other cellular mediators, nutrients, antibiotics, antiinflammatories, and other pharmaceuticals. Although not limiting, some suitable cellular growth factors, depending upon the cell type to be encapsulated in either the hydrogel of the same or adjacent hydrogel layer, include heparin binding growth factor (HBGF), transforming growth factor (TGFα or TGFβ), alpha fibroblastic growth factor (FGF), epidermal growth factor (EGF), vascular endothelium growth factor (VEGF), various angiogenic factors, nerve growth factor (NGF) and muscle morphologic growth factor.

In addition, the hydrogel solution optionally includes a suitable non-toxic polymerization initiator, mixed thoroughly to make a final concentration of 0.05% w/v. When PEGDA or PEODA are selected as the polymers, the polymerization initiator is preferably added and selected to be the photoinitiator Igracure 2959 (commercially available from Ciba Specialty Chemicals Corp., Tarrytown, N.Y.), although other suitable photoinitiators can be used.

While photopolymerizable PEGDA and PEODA are among the preferred polymers for making hydrogels in accordance with the present invention, other suitable hydrophilic polymers can be used. Suitable hydrophilic polymers include synthetic polymers such as partially or filly hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll® polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. This list of photopolymerizable mixtures is meant to be illustrative and not exhaustive. For example, other photopolymerizable mixtures suitable for application in the present invention are described in U.S. Pat. No. 6,224,893 B1, which has been incorporated herein by reference.

Likewise, while the preferred photoinitiator is Igracure 2959, various other photoinitiators can be used instead. For example, HPK, which is commercially available from Polysciences, is another suitable photoinitiator. In addition, various dyes and an amine catalyst are known to form an active species when exposed to external radiation. Specifically, light absorption by the dye causes the dye to assume a triplet state, which subsequently reacts with the amine to form the active species that initiates polymerization. Typically, polymerization can be initiated by irradiation with light at a wavelength of between about 200-700 nm, most preferably in the long wavelength ultraviolet range or visible range, 320 nm or higher, and most preferably between about 365 and 514 nm.

Numerous dyes can be used for photopolymerization, and these include erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable cocatalysts include amines such as N-methyl diethanolamine, N,N-dimethyl benzylamine, triethanol amine, triethylamine, dibenzyl amine, N-benzylethanolamine, N-isopropyl benzylamine. Triethanolamine is a preferred cocatalyst with one of these dyes. Photopolymerization of these polymer solutions is based on the discovery that combinations of polymers and photoinitiators (in a concentration not toxic to the cells, less than 0.1% by weight, more preferably between 0.05 and 0.01% by weight percent initiator) will crosslink upon exposure to light equivalent to between one and 3 mWatts/cm².

While photopolymers are preferred for making the hydrogels, because it is convenient to control polymerization using external radiation supplied through a surgical scope, the present invention can be practiced using other polymer materials and polymerization initiators. Examples of other materials which can be used to form a hydrogel include (a) modified alginates, (b) polysaccharides (e.g. gellan cum and carrageenans) which gel by exposure to monovalent cations, (c) polysaccharides (e.g., hyaluronic acid) that are very viscous liquids or are thiotropic and form a gel over time by the slow evolution of structure, and (d) polymeric hydrogel precursors (e.g., polyethylene oxide-polypropylene glycol block copolymers and proteins). U.S. Pat. No. 6,224,893 B1 provides a detailed description of the various polymers, and the chemical properties of such polymers, that are suitable for making hydrogels in accordance with the present invention, and this patent is incorporated herein by reference in its entirety.

The list of hydrogels described in U.S. Pat. No. 6,224,893 B1 are reproduced below. The polymerizable agent of the present invention may comprise monomers, macromers, oligomers, polymers, or a mixture thereof. The polymer compositions can consist solely of covalently crosslinkable polymers, or blends of covalently and ionically crosslinkable or hydrophilic polymers.

Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

Examples of materials that can be used to form a hydrogel include modified alginates. Alginate is a carbohydrate polymer isolated from seaweed, which can be crosslinked to form a hydrogel by exposure to a divalent cation such as calcium, as described, for example in WO 94/25080, the disclosure of which is incorporated herein by reference. Alginate is ionically crosslinked in the presence of divalent cations, in water, at room temperature, to form a hydrogel matrix. Modified alginate derivatives may be synthesized which have an improved ability to form hydrogels. The use of alginate as the starting material is advantageous because it is available from more than one source, and is available in good purity and characterization. As used herein, the term “modified alginates” refers to chemically modified alginates with modified hydrogel properties. Naturally occurring alginate may be chemically modified to produce alginate polymer derivatives that degrade more quickly. For example, alginate may be chemically cleaved to produce smaller blocks of gellable oligosaccharide blocks and a linear copolymer may be formed with another preselected moiety, e.g. lactic acid or epsilon-caprolactone. The resulting polymer includes alginate blocks which permit ionically catalyzed gelling, and oligoester blocks which produce more rapid degradation depending on the synthetic design. Alternatively, alginate polymers may be used wherein the ratio of mannuronic acid to guluronic acid does not produce a film gel, which are derivatized with hydrophobic, water-labile chains, e.g., oligomers of epsilon-caprolactone. The hydrophobic interactions induce gelation, until they degrade in the body.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Polysaccharides that are very viscous liquids or are thixotropic, and form a gel over time by the slow evolution of structure, are also useful. For example, hyaluronic acid, which forms an injectable gel with a consistency like a hair gel, may be utilized. Modified hyaluronic acid derivatives are particularly useful. As used herein, the term “hyaluronic acids” refers to natural and chemically modified hyaluronic acids. Modified hyaluronic acids may be designed and synthesized with preselected chemical modifications to adjust the rate and degree of crosslinking and biodegradation. For example, modified hyaluronic acids may be designed and synthesized which are esterified with a relatively hydrophobic group such as propionic acid or benzylic acid to render the polymer more hydrophobic and gel-forming, or which are grafted with amines to promote electrostatic self-assembly. Modified hyaluronic acids thus may be synthesized which are injectable, in that they flow under stress, but maintain a gel-like structure when not under stress. Hyaluronic acid and hyaluronic derivatives are available from Genzyme, Cambridge, Mass. and Fidia, Italy.

Other polymeric hydrogel precursors include polyethylene oxide-polypropylene glycol block copolymers such as Pluronics™ or Tetronics™, which are crosslinked by hydrogen bonding and/or by a temperature change, as described in Steinleitner et al., Obstetrics & Gynecology, 77:48-52 (1991); and Steinleitner et al., Fertility and Sterility, 57:305-308 (1992). Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin.

Suitable ionically crosslinkable groups include phenols, amines, imines, amides, carboxylic acids, sulfonic acids and phosphate groups. Aliphatic hydroxy groups are not considered to be reactive groups for the chemistry disclosed herein. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

The preferred anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO₃H groups such as sulfonated polystyrene, and polystyrene with carboxylic acid groups. These polymers can be modified to contain active species polymerizable groups and/or ionically crosslinkable groups. Methods for modifying hydrophilic polymers to include these groups are well known to those of skill in the art.

The polymers may be intrinsically biodegradable, but are preferably of low biodegradability (for predictability of dissolution) but of sufficiently low molecular weight to allow excretion. The maximum molecular weight to allow excretion in human beings (or other species in which use is intended) will vary with polymer type, but will often be about 20,000 daltons or below. Usable, but less preferable for general use because of intrinsic biodegradability, are water-soluble natural polymers and synthetic equivalents or derivatives, including polypeptides, polynucleotides, and degradable polysaccharides.

The polymers can be a single block with a molecular weight of at least 600, preferably 2000 or more, and more preferably at least 3000. Alternatively, the polymers can include can be two or more water-soluble blocks which are joined by other groups. Such joining groups can include biodegradable linkages, polymerizable linkages, or both. For example, an unsaturated dicarboxylic acid, such as maleic, fumaric, or aconitic acid, can be esterified with hydrophilic polymers containing hydroxy groups, such as polyethylene glycols, or amidated with hydrophilic polymers containing amine groups, such as poloxamines.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized, as disclosed in WO 93/17669, the disclosure of which is incorporated herein by reference. In this embodiment, water soluble macromers that include at least one water soluble region, a biodegradable region, and at least two free radical-polymerizable regions, are provided. The macromers are polymerized by exposure of the polymerizable regions to free radicals generated, for example, by photosensitive chemicals and or light. Examples of these macromers are PEG-oligolactyl-acrylates, wherein the acrylate groups are polymerized using radical initiating systems, such as an eosin dye, or by brief exposure to ultraviolet or visible light. Additionally, water soluble polymers which include cinnamoyl groups which may be photochemically crosslinked may be utilized, as disclosed in Matsuda et al., ASAID Trans., 38:154-157 (1992).

The term “active species polymerizable group” is defined as a reactive functional group that has the capacity to form additional covalent bonds resulting in polymer interlinking upon exposure to active species. Active species include free radicals, cations, and anions. Suitable free radical polymerizable groups include ethylenically unsaturated groups (i.e., vinyl groups) such as vinyl ethers, allyl groups, unsaturated monocarboxylic acids, unsaturated dicarboxylic acids, and unsaturated tricarboxylic acids. Unsaturated monocarboxylic acids include acrylic acid, methacrylic acid and crotonic acid. Unsaturated dicarboxylic acids include maleic, flimaric, itaconic, mesaconic or citraconic acid. In one embodiment, the active species polymerizable groups are preferably located at one or more ends of the hydrophilic polymer. In another embodiment, the active species polymerizable groups are located within a block copolymer with one or more hydrophilic polymers forming the individual blocks. The preferred polymerizable groups are acrylates, diacrylates, oligoacrylates, dimethacrylates, oligomethacrylates, and other biologically acceptable photopolymerizable groups. Acrylates are the most preferred active species polymerizable group.

In general, the polymers are at least partially soluble in aqueous solutions, such as water, buffered salt solutions, or aqueous alcohol solutions. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring and synthetic polymers may be modified using chemical reactions available in the art and described, for example, in March, “Advanced Organic Chemistry,” 4th Edition, 1992, Wiley-Interscience Publication, New York.

Preferably, the hydrophilic polymers that include active species or crosslinkable groups include at least 1.02 polymerizable or crosslinkable groups on average, and, more preferably, each includes two or more polymerizable or crosslinkable groups on average. Because each polymerizable group will polymerize into a chain, crosslinked hydrogels can be produced using only slightly more than one reactive group per polymer (i.e., about 1.02 polymerizable groups on average). However, higher percentages are preferable, and excellent gels can be obtained in polymer mixtures in which most or all of the molecules have two or more reactive double bonds. Poloxamines, an example of a hydrophilic polymer, have four arms and thus may readily be modified to include four polymerizable groups.

Additional hydrogels suitable for practicing the present invention are described in U.S. Pat. No. 5,567,435 to Hubbell et al., which is also incorporated herein by reference in its entirety.

Immediately prior to encapsulation, the target cells for encapsulation are suspended from a cell pellet form using the hydrogel solution (also referred to as the polymer solution). Specifically, the polymer solution is gently and thoroughly mixed with the cell pellet containing the target cells in an amount to make a homogenous suspension having a cellular concentration of about 20 million cells/cc. It is noted that a separate polymer-cell suspension must be made for each layer of the multi-layered tissue construct containing cells. For example, if a bi-layered tissue construct is being engineered, with each layer having a different cell type, then two different polymer-cell suspensions must be made. Each suspension preferably uses the same hydrogel solution and the same polymerization initiator; however, the cell types suspended in the hydrogel solution will generally be different. Likewise, when three layers are to be created, with each layer having cells, then three different polymer-cell suspensions need to be prepared, and so on.

While it is preferable to make a multi-layered tissue construct using the same hydrogel material for each layer, the present invention can be practiced by using different hydrogel materials for one or more of the layers. For example, it is within the scope of the present invention to make a multi-layered tissue construct having a hydrogel layer formed by polymerizing PEODA and another hydrogel layer formed by polymerizing a modified alginate derivative. This example is, of course non-limiting, and other hydrogel polymers could be layered together to form a tissue construct within the scope of the present invention.

Layer Formation/Cell Encapsulation Steps

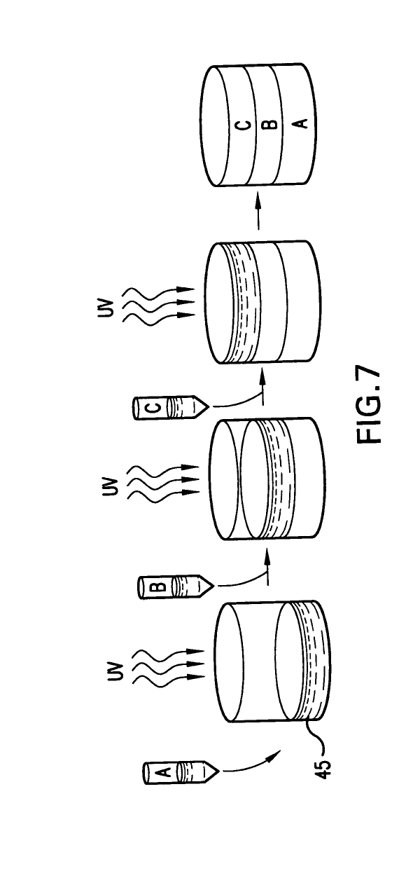

The third step 40 in the method for making, or creating, a multi-layered tissue construct in accordance with the present invention involves placing a predetermined volume of a first polymer-cell suspension A in the target space as shown in FIG. 7. In this case, the target space is illustrated as a cavity in a mold 45. However, the target space can also be a cavity present in tissue.

The fourth step 50 involves partially gelling the first polymer-cell suspension A by providing a polymerization initiator to initiate crosslinking or by allowing the polymer-cell suspension A to polymerize on its own. FIG. 7 illustrates a preferred embodiment of the present invention, wherein suspension A is a photopolymer-cell suspension containing a photoinitiator. In this case, exposing the photopolymer-cell suspension A to an external radiation source converts the photoinitiator to an active species and polymer crosslinking is initiated in a controlled fashion. Preferably, when practicing this embodiment the external radiation source is a UVA lamp having a wavelength of 200 nm or greater so as to expose the suspension to a radiation intensity of about 1-4 mW/cm². Furthermore, when practicing this embodiment, the radiation exposure time is about 3-5 minutes depending upon the degree of partial gelling desired.

Next, the method moves to the decision point 60. However, because there is only one layer formed so far, the method returns to step 40, wherein a predetermined volume of a second polymer-cell suspension B is placed in the target space. When practicing this embodiment, as shown in FIG. 7, suspension B is a photopolymer-cell suspension containing a photoinitiator and crosslinking of the photopolymer is initiated and controlled by exposing both suspension A and suspension B to WVA irradiation as described in step 50 for about 2-3 minutes in order to partially gel the second polymer-cell suspension B.

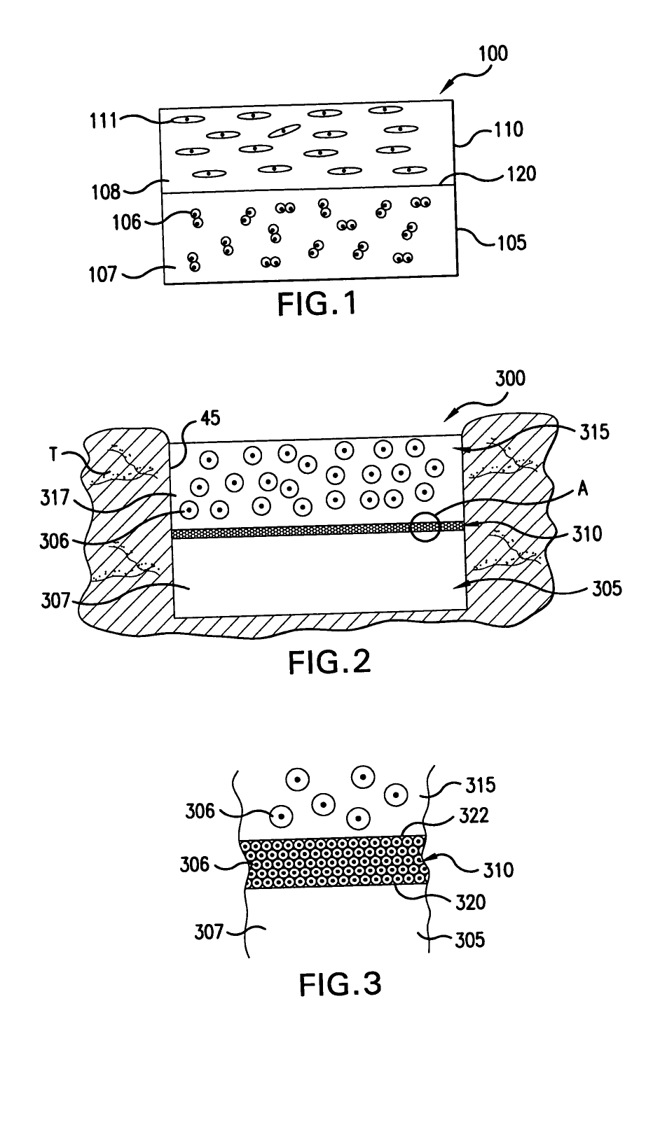

The method now returns to decision point 60. When making a two-layered tissue construct 100, such as shown in FIG. 1, the answer to the decision point 60 would be “no” at this point and the method is progressed to step 70. Step 70 involves ensuring that all hydrogel layers of the tissue construct have completely gelled either by passively allowing the partially gelled layers more time to crosslink, or by actively controlling additional crosslinking.

When practicing this embodiment, additional crosslinking of the photopolymer can be actively controlled by exposing all layers of the partially gelled tissue construct to additional radiation to completely gel the multi-layered tissue construct. So, in the case of the two-layered tissue construct 100, the hydrogel layers 105, 110 formed by polymerizing suspension A and suspension B, respectively, are both irradiated with the external UV radiation for an additional time period, generally about 2-3 minutes, to ensure that complete gelling of each layer has occurred.

Optionally, step 70 is followed by step 80, wherein the completely gelled multi-layered tissue construct 100 is removed from the space used to create the construct and placed in a container provided with a complete incubation media, such as Dulbeco's Modified Eagle's Medium with or without other additives. The multi-layered tissue construct is then incubated until ready for transplant, which may be several weeks. Those skilled in the art would realize that step 80 is performed only when the tissue construct is created in vitro (i.e., in a mold). However, when the tissue construct is created in vivo and the space used for making the tissue construct is a cavity in tissue, then step 80 would not apply. Specifically, when creating the multi-layered tissue construct directly in living tissue in the host recipient, the tissue construct is implanted directly into the host while it is being made so there can be no incubation step for the tissue construct prior to transplant.

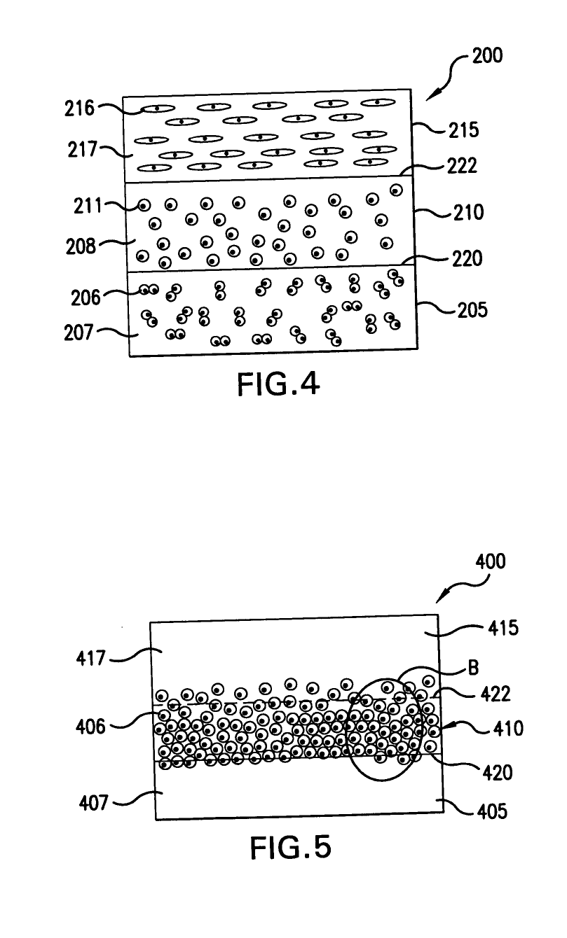

In the case where a multi-layered tissue construct having three or more layers is desired, the method for making, or creating, a multi-layer tissue construct in accordance with the present invention would proceed differently at decision point 60. Specifically, after the polymer-cell suspension A and the polymer-cell suspension B have been partially gelled in the space, additional layers are added be repeating steps 40 to 60. For example, when creating a three-layered tissue construct 200, as shown in FIG. 4, the method in accordance with the present invention would have progressed at this point from decision point 60 (with an answer of “yes”) to step 40. Then a predetermined volume of polymer-cell suspension C is placed in the target space.

When practicing the this embodiment, suspension C is a photopolymer-cell suspension containing a photoinitiator and, as shown in FIG. 7, crosslinking of the photopolymer is controlled by exposing the photopolymer-cell suspension C to external radiation. Thus, suspension A, suspension B and suspension C are exposed to UV irradiation, as described in step 50, for about 2-3 minutes in order to partially gel the third photopolymer-cell suspension C.

The method returns again to decision point 60. When making a three-layered tissue construct 200, such as shown in FIG. 4, the answer to the decision point 60 would be “no” at this point and the method is progressed to step 70. Step 70 involves ensuring that all hydrogel layers have completely gelled by either passively allowing the suspensions more time to crosslink, or by actively controlling additional crosslinking.

When practicing this embodiment, additional crosslinking is actively controlled by exposing all layers of the partially gelled tissue construct to additional radiation to completely gel the multi-layered tissue construct. So, in the case of the three-layered tissue construct 200, the hydrogel layers 205, 210, 215 formed by photopolymerizing suspension A, suspension B, and suspension C, respectively, are irradiated with the external UV radiation for an additional time period, generally about 2-3 minutes, to ensure that complete gelling of each layer has occurred.

As discussed above, in the case where the target space is a cavity formed in tissue, the method ends here when making a three-layered tissue construct. However, when using a mold cavity to provide the target space, the method can optionally include step 80, which involves removing the multi-layered tissue construct 200 from the mold and placing it in a complete media for further incubation until ready for transplantation.

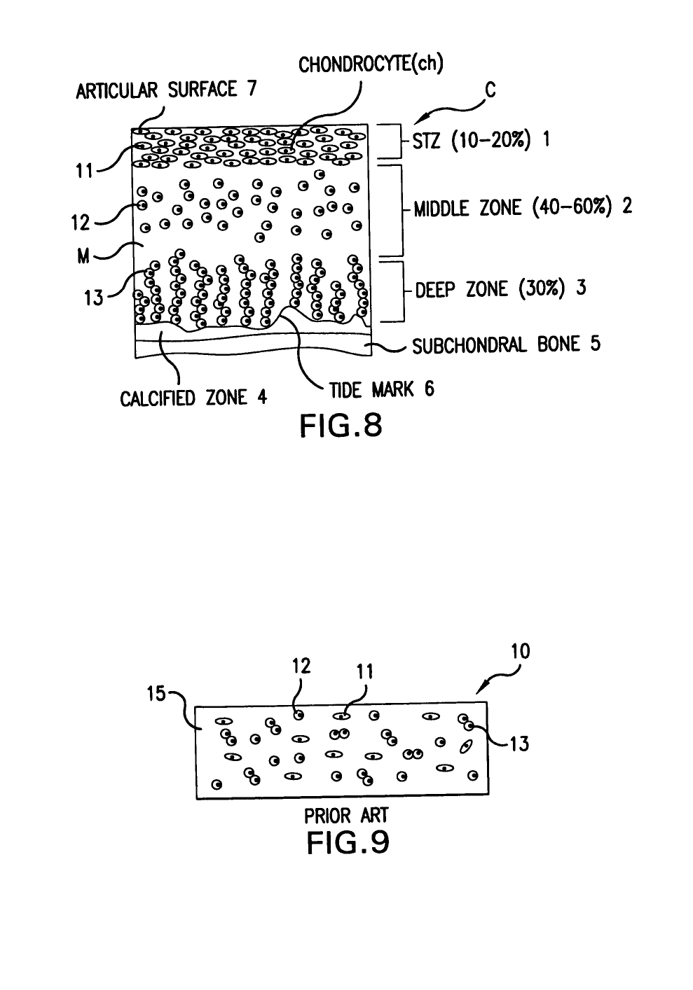

Those skilled in the art would realize the method outlined in FIG. 6 can be used to create multi-layered tissue constructs having more than three layers by reiterating through steps 40, 50 and 60 until the desired number of layers are made. In step 30, it is necessary to prepare the same number of polymer-cell suspensions as would correspond to the number of hydrogel layers containing distinctly different cell types, assuming each layer contains cells. For example, in FIG. 8, the cartilage-bone interface is illustrated as having five zones: superficial STZ zone 1 containing reserve chondrocytes, middle zone 2 containing proliferating chondrocytes, deep zone 3 containing hypertrophying chondrocytes, calcified zone 4 containing calcifying cartilage, and subchondral bone 5 containing osteoblasts. In accordance with the present invention, the method outlined in FIG. 6 can be used to create a five-layered tissue construct, wherein five different photopolymer-cell suspensions would be prepared in step 30 and steps 40-60 reiterated until a five-layered tissue construct is made. Then, the method would progress to completely gelling the multi-layered tissue construct in step 70, optionally followed by the incubation step 80 if the construct 200 was created in a target space in a mold.

Description of the Structure of Multi-Layered Tissue Constructs

The method for making, or creating, a multi-layer tissue construct in accordance with the present invention has been generally described above in detail. Next, the structure of various multi-layered tissue constructs will be generally described in detail before describing particular non-limiting illustrative embodiments.

FIG. 1 shows a multi-layered tissue construct 100 in accordance with the present invention that has two layers 105 and 110. The first layer 105 includes cells 106 predominately of a first cell type encapsulated in the hydrogel 107. Hydrogel 107 is the polymerized network formed from polymerization of one of the suitable polymers described above and has a high water content. The second layer 110 includes cells 111 predominately of a second cell type and the hydrogel 108. Preferably, hydrogel 107 and hydrogel 108 are the same material. However, the present invention can be practiced wherein hydrogel 108 is the polymerized network formed from polymerization of another one of the suitable polymers described above and has a high water content, wherein the polymer used to make hydrogel 108 is different from the polymer used to make hydrogel 107. Generally, the first cell type 106 and the second cell type 111 are different cell types.

In the context of this disclosure, cells are considered to be the “same type” if they have the same phenotype, which means they have the same gene expression and/or morphology. Gene expression in this context includes the expression of cell surface proteins and/or protein secretion. Consequently, cells are considered to by “different types” when they are derived from different tissue types (e.g., cartilage versus bone), the cells are derived from different embryonal origin (e.g., ectodermal versus mesodermal versus endodermal origin), the cells have a significantly different degree of maturation (e.g., stem cells versus partially differentiated cells versus completely differentiated cells), and the cells that are otherwise similar except for gene expression and morphology (e.g., superficial chondrocytes versus deep chondrocytes). To illustrate this point, for example, cells that are deep zone chondrocytes are “not different” from one another because they all come from the same type of tissue (i.e., cartilage), have the same embryonal origin, have the same degree of maturation (i.e., are mature cells), and otherwise share the same gene expression and morphology as other chondrocytes in the deep zone of cartilage.

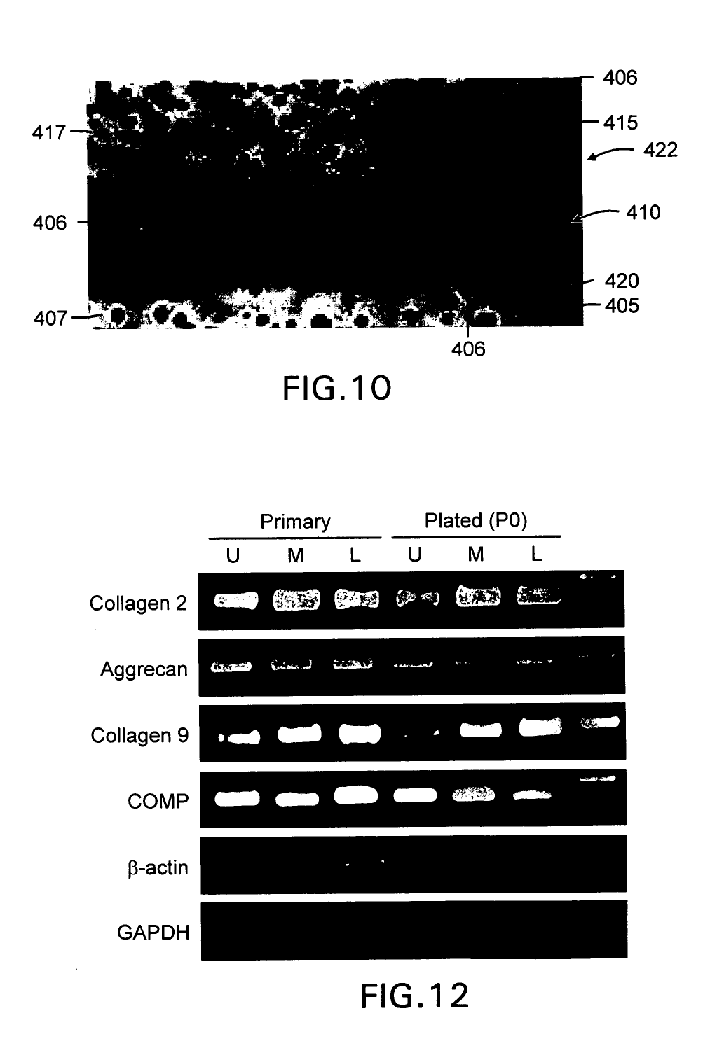

The first layer 105 of tissue construct 100 is connected to the second layer 110 through a transition zone 120. The transition zone 120 was formed when the second layer 110 was partially gelled on the already partially gelled first layer 105. The transition zone 120 can be fairly abrupt or there can be a smooth transition depending upon the degree of partial gelling of the first layer 105 when the second layer 110 was formed. Although representing a different embodiment of the present invention, FIG. 10 illustrates the meaning of what is an abrupt transition zone and what is a smooth transition zone.

As shown in FIG. 10, an abrupt transition zone occurs when the supporting hydrogel layer is mostly gelled before the addition of another layer of cells or a polymer-cell suspension. Under these conditions, there is very little mixing of the cells from the added layer into the mostly gelled layer. On the other hand, when there is very little or no gelling of the supporting hydrogel layer before the addition of the other layer of cells or polymer-cell suspension, the result is that many of the cells in the supporting layer mix into the added layer thereby creating a “smooth transition” as shown in FIG. 10.

Because of the high water content of hydrogels 107 and 108 in the two connected layers 105, 110, the transition zone 120 is permeable to products of cellular metabolism in both layers. Therefore, cellular mediators produced by cells 106 of the first type should be able to cross the transition zone 120 and affect cells 111 of the second cell type. Likewise, cellular mediators produced by cells 111 of the second cell type should be able to cross the transition zone 120 and affect cells 106 of the first cell type. This permeable feature of the transition zone 120 is important to preserve interaction between different cell types organized in different zones and to mimic the environment in real tissues. In some embodiments in accordance with the present invention, one of the hydrogel layers 105 and 110 can be formed from polymer suspensions that include a substance, such as a nutrient, cellular mediator or pharmaceutical, instead of, or in addition to, cells to be encapsulated.

While the cell types 106 and 111 are not particularly limited to any particular combination of cell types, in one particular embodiment in accordance with the present invention, one of the cell types 106 or 111 is a stem cell. For example, when one of the cell types 106 is a mesenchymal stem cell and the other cell type 111 is an “educator cell,” such as an articular cartilage chondrocyte, the mesenchymal stem cell can differentiate into a bone producing cell. This embodiment is useful because the educator cell “teaches” or induces the mesenchymal stem cell to differentiate into a cell type that produces bone, which can be used in treating defects in bone. In another useful embodiment, for example, when one of the cell types 106 is a pluri-potent or multi-potent embryonic stem cell and the other cell type 111 is an “educator cell,” such as a chondrocyte, the embryonic stem cell differentiates into a cartilage matrix producing cell. This embodiment is useful because the educator cell “teaches” or induces the pluri-potent embryonic stem cell to differentiate into a cell type that produces cartilage matrix, which can be used in treating defects in cartilage.

FIG. 4 shows a multi-layered tissue construct 200 in accordance with the present invention that has three layers 205, 210 and 215. The first layer 205 includes cells 206 predominately of a first cell type encapsulated in the hydrogel 207. Hydrogel 207 is the polymerized network formed from polymerization of one of the suitable polymers described above and has a high water content. The second layer 210 includes cells 211 predominately of a second cell type and the hydrogel 208. The third layer 215 includes cells 216 predominately of a third cell type and the hydrogel 217. Preferably, hydrogels 207, 208 and 217 are the same material. However, the present invention can be practiced wherein hydrogel 208 is the polymerized network formed from polymerization of another one of the suitable polymers described above and has a high water content, wherein the polymer used to make hydrogel 208 is different from the polymer used to make hydrogel 207 and/or hydrogel 217. Likewise, the present invention can be practiced wherein hydrogel 217 is the polymerized network formed from polymerization of yet another one of the suitable polymers described above and has a high water content, wherein the polymer used to make hydrogel 217 is different from the polymer used to make hydrogel 207 and/or hydrogel 208. In other words, all of the hydrogel layers can be made of the same hydrogel material, or all of the hydrogel layers can be made from different hydrogel materials, or some, but not all, of the hydrogel layers can be made of the same hydrogel material.

Generally, the first cell type 206, the second cell type 211 and the third cell type 216 are different cell types. However, the present invention can be practiced where some of the layers include the same cell types, although these would preferably not be contiguous layers. In addition, when practicing embodiments in accordance with the present invention that have three or more hydrogel layers, some of the hydrogel layers may be formed from a polymer suspension that does not contain any cells. Under these conditions, the hydrogel formed from a polymer suspension that does not contain cells would be a “cell-less” (i.e., may be free of cells) hydrogel layer to the degree that some cells may spill over the transition zones.

The first layer 205 of tissue construct 200 is connected to the second layer 210 through a transition zone 220. The transition zone 220 was formed when the second layer 210 was partially gelled on the already partially gelled first layer 205. The transition zone 220 can be fairly abrupt or there can be a smooth transition depending upon the degree of partial gelling of the first layer 205 when the second layer 210 was formed. The second layer 210 is connected to the third layer 215 through a transition zone 222. The transition zone 222 was formed when the third layer 215 was partially gelled on the already partially gelled second layer 210. The transition zone 222 can be fairly abrupt or there can be a smooth transition depending upon the degree of partial gelling of the second layer 210 when the third layer 215 was formed.

As discussed above, transition zones 220 and 222 are permeable so nutrients and products of cellular metabolism can diffuse between the layers. In some embodiments in accordance with the present invention, one or more of the hydrogel layers 205, 210, 215 can be formed from polymer suspensions that include a bioactive additive, such as a nutrient, a cellular mediator, growth factors, compounds which induce cellular differentiation, a bioactive polymer, a gene vector, or a pharmaceutical (i.e., antibiotics, antiinflammatories, etc.), instead of, or in addition to, cells to be encapsulated. In the case where a layer does not include cells, the bioactive additive is mixed in with the polymer solution. The bioactive additive can be added to the polymer solution or the polymer-cell suspension during synthesis. In addition, the bioactive additive, when added to the polymer solution or to the polymer-cell suspension, can be contained in a delivery vehicle, such as a microsphere, liposomes, and the like.

In accordance with the present invention, the hydrogel layers can also include other additives that promote structural integrity and strength. These additives are mixed into the polymer solution or the polymer-cell suspension during synthesis. Examples of other additives to improve the mechanical properties of the hydrogels include hyaluronic acid and hydroxyapatite.

FIG. 3 illustrates another embodiment in accordance with the present invention, which is a multi-layered tissue construct 300 that has three layers 305, 310 and 315, wherein the middle layer 310 is formed differently than in step 30 to 50 of the above described method. Specifically, the method outlined in FIG. 6 is modified so that (a) the suspension corresponding to base layer 305 includes polymer and no cells, and (b) the suspension corresponding to the middle layer 320 is comprised of cells and no polymer. However, the suspension corresponding to the upper layer 315 is prepared to include both cells and polymer. In addition, the cells 306 in layers 310 and 315 are the same type of cells, which are preferably some type of stem cell.

Under these conditions, when the first layer 305 is formed it is basically a “cell-less” hydrogel layer 307. However, as evident from FIG. 10, some cells 306 will become encapsulated in the first layer 305 near the transition zone 320. Hydrogel 307 is the polymerized network formed from polymerization of one of the suitable polymers described above and has a high water content. The second layer 310 includes cells 306 in a densely packed layer. As discussed above, the suspension used to make the second layer 310 did not include any polymer. However, when this polymer-less suspension is placed upon first layer 305 some of the unpolymerized polymer of hydrogel 307 may mix into the suspension that will form second layer 310. Because there is relatively little polymer in the second layer at this time, the suspension corresponding to the third layer 315 is placed onto the second layer without performing a distinct partial gelling step 50.

To a greater degree, when the polymer-cell suspension corresponding to the third layer 315 is placed on the second layer 310, uncrosslinked polymer is free to mix into the second layer. Consequently, while the second layer 310 includes a very high cellular density, it will also include some polymer from the third layer 315 and possibly some polymer from the first layer 310. Subsequently, the partial gelling step 50 is applied simultaneously to both the second layer 310 and the third layer 315. When gelling of all layers has been completed in step 70, a transition zone 320 will have formed between the first layer 305 and the second layer 310, and a transition zone 322 will have formed between the second layer 310 and the third layer 315 as schematically illustrated in FIG. 3, which is a magnified view of region A in FIG. 2.

The third layer 315 includes cells 306 and the hydrogel 317. Preferably, hydrogels 307 and 317 are the same material; however, the multi-layered tissue construct 300 of the present invention can be practiced wherein hydrogel 308 is the polymerized network formed from polymerization of a polymer that is different from the polymer used to make hydrogel 317. Thus, while the top layer 315 and the base layer 305 are preferably made of the same hydrogel material, these two hydrogel layers could be made using different polymers, and/or these two layers could have different additives without departing from the scope and spirit of the invention.

While the multi-layered tissue construct 300 can be engineered in a mold, this construct in particular can be used to treat defects in tissue. When used in this manner, the cells 306 are preferably stem cells that will differentiate into a desired cell type while growing in a defect (i.e., cavity 45 shown in FIG. 2) in a tissue T.

FIG. 5 illustrates another embodiment in accordance with the present invention, which is a multi-layered tissue construct 400 that has three layers 405, 410 and 415, wherein the middle layer 410 is formed differently than in steps 30 to 50 of the above described method. Specifically, the method outlined in FIG. 6 is modified so that (a) the suspension corresponding to base layer 405 and top layer 415 includes polymer and no cells, and (b) the suspension corresponding to the middle layer 410 is comprised of cells and no polymer. Consequently, the cells 406 is the only cell type in this embodiment. While not limited to any particular cell type, multi-layered tissue construct 400 is preferably made using some type of stem cell.

Under the conditions described above, when the first layer 405 is formed it is basically a “cell-less” hydrogel layer 407. However, shown in FIG. 10, some cells 406 will become encapsulated in the first layer 405 near the transition zone 420. Hydrogel 407 is the polymerized network formed from polymerization of one of the suitable polymers described above and has a high water content. The second layer 410 includes cells 406 in a densely packed layer. As discussed above, the suspension used to make the second layer 410 did not include any polymer. However, when this polymer-less suspension is placed upon first layer 405 some of the unpolymerized polymer of hydrogel 407 may mix into the suspension that will form second layer 310. Because there is relatively little polymer in the second layer at this time, the suspension corresponding to the third layer 415 is placed onto the second layer without performing a distinct partial gelling step 50.

To a greater degree, when the polymer-cell suspension corresponding to the third layer 415 is placed on the second layer 410, uncrosslinked polymer is free to mix into the second layer. Consequently, while the second layer 410 includes a very high cellular density, it will also include some polymer mixed in from the third layer 415 and possibly some polymer mixed in from the first layer 410. Subsequently, the partial gelling step 50 is applied simultaneously to both the second layer 410 and the third layer 415. When gelling of all layers has been completed in step 70, a relatively abrupt, or sharp, transition zone 420 will have formed between the first layer 405 and the second layer 410, and a relatively smooth, or smeared, transition zone 422 will have formed between the second layer 410 and the third layer 415 as illustrated in the photograph in FIG. 3, which is a magnified view of region B shown in the schematically drawn FIG. 5.

Those skilled in the art will realize that because base layer 405 and top layer 415 were made from cell-less polymer suspensions, and that all cells 406 in these layers originated from the cell suspension used to make middle layer 410. Furthermore, it is easier to appreciate from FIGS. 5 and 10 how cells from the middle layer 410 become encapsulated into the adjacent layers, although to a different degree. This same phenomenon occurs when making the other embodiments, although it is more difficult to appreciate when adjacent hydrogel layers are made from polymer-cell suspensions.

In addition, while the top layer 415 and the base layer 405 are preferably made of the same hydrogel material, these two hydrogel layers could be made using different polymers, and/or these two layers could have different additives without departing from the scope and spirit of the invention.

While the present invention, and its main modifications, have been described in detail, several specific illustrative examples highlighting certain advantages are described below.

### ILLUSTRATIVE EXAMPLE 1

### Multi-Layered Tissue Construct Encapsulating Chondrocytes from Three Zones of Articular Cartilage

In this illustrative example, a three-layered tissue construct, such as shown in FIG. 4, is created using a photopolymer, a photoinitiator and UVA radiation to effect crosslinking and hydrogel formation, and the encapsulated cells are chondrocytes harvested from three different tissue zones in mammalian articular cartilage. The present three-layered tissue construct, while formed in the cavity of a mold, is suitable for transplantation and could have been engineered in situ directly in the cavity of an articular joint defect.

First, chondrocytes corresponding to the three different cell types to be encapsulated in the different hydrogel layers were harvested. Cartilage slices were taken from the patellofemoral groove and femoral condyles of 6 legs from three 5-8 week old calves. To obtain cartilage blocks with similar shape, only central areas were removed from the patellofemoral groove, medial femoral condyle, and lateral femoral condyle. In order to facilitate defining the three zones, cartilage was taken en bloc from the subchondral bone. The thickness of the cartilage block ranged from 2 to 6 mm depending on the joint area. To minimize the contamination by cells from adjacent zones, only the top 10%, central 10%, and bottom 10% were taken for the upper, middle, and lower zones, respectively. Briefly, the top 10% (200-600 μm) was first taken from the cartilage block using a surgical blade. After the following 30% was discarded, the next 10% (200-600 μm) was taken for the middle zone. After the following 30% and the most bottom 10% including remaining subchondral bone were discarded, the bottom 10% (200-600 μm) was harvested for the lower zone.

Phenotypic Characterization of Harvested Chondrocytes

To confirm that cartilage slices were obtained from the specific zone, histologic evaluation of the cartilage taken en bloc and cartilage slices from three layers was performed. Formalin fixed, paraffin embedded specimens were sectioned and stained with Safranin-O/Fast Green and Masson's trichrome using standard histological procedure.

Histologic evaluation allowed visual confirmation that cartilage slices had been obtained from the upper (superficial STZ), middle and lower (deep) zones 1, 2, 3 of the cartilage block. The upper zone had the highest cellularity, followed by the middle zone and lower zone. Cells of the upper zone were smaller than cells of the middle and lower zones. Cells along the articular surface of the upper zone showed flattened or ellipsoid-shaped morphology and parallel arrangement with the articular surface. The intensity of Safranin-O staining, indicating proteoglycan content, was the highest in the lower zone followed by the middle and upper zones. The intensity of Masson's trichrome staining, directly related to collagen content, was the highest in the middle zone followed by the upper and lower zones.

Biochemical compositions of the excised cartilage slices were determined by DNA assay, glycosaminoglycan (GAG) assay, and collagen assay, which provide various properties for describing the phenotype of the chondrocyte cell type in each one of the three zones. Wet weights (ww) and dry weights (dw) were obtained from the cartilage slices (n=9, from three different animals) before and after 48 hours of lyophilization. The dried specimens were digested in 1 ml of papain solution (125 μg/ml Papain, Worthington Biomedical Corporation, Lakewood, N.J.), 100 mM phosphate buffer, 10 mM cysteine, 10 mM EDTA, pH 6.3] for 18 hours at 60° C. The DNA content (ng of DNA/mg dw of the cartilage slice) was determined using Hoechst 33258. Glycosaminoglycan (GAG) content was estimated by chondroitin sulfate using dimethylmethylene blue dye. Total collagen content was determined by measuring the hydroxyproline content of the specimens after acid hydrolysis and reaction with p-dimethylaminobenzaldehyde and chloramine-T using 0.1 as the ratio of hydroxyproline to collagen. All biochemical results are presented as means and standard deviations (n=9).

Results of the biochemical assays of cartilage slices from different layers were consistent with the histologic findings. The water content was the highest in the upper zone and was over 80%, while the water content of the other two layers was below 80%, and the difference in the water content between the upper zone compared to each one of the other two zones was significant (p<0.01). The upper zone also had the highest DNA content ranging between 1.5-2 μg/mg wet weight, which was in line with the highest cellularity observed in the histologic examination. Both the middle and lower zones had significantly lower (p<0.01) DNA content, which was about 1 μg/mg wet weight or less. Glycosaminoglycan (GAG) content of the lower zone was the greatest at about 60% in dry weight, followed by the middle and upper zones that each had about 42 and 38% in dry weight, respectively. Each zone had a GAG content that was significantly different from the GAG contents of the other two zones (p<0.01). The middle zone had the highest collagen content at about 78% in dry weight, followed by the upper and lower zones at about 70% and 59% in dry weight respectively. The difference in collagen was more significant between the middle zone and the lower zone (p<0.01) than it was between the middle zone and the upper zone (p<0.05).

To isolate chondrocytes, the cartilage pieces were incubated in Dulbeco's Modified Eagle's Medium (DMEM, GIBCO, Grand Island, N.Y., U.S.A.) containing 0.2% collagenase (Worthington Biochemical Corporation, Lakewood, N.J., U.S.A.) and 5% fetal bovine serum (GIBCO) for 14-16 hours at 37° C. and 5% CO₂ . The resulting cell suspensions were then filtered through 70 μm nylon filters (Cell Strainer; Falcon, Franklin Lakes, N.J., U.S.A.) and washed three times with Phosphate Buffered Saline (PBS) containing 100 U/ml penicillin and 100 μg/ml streptomycin. The number and sizes of the isolated cells were then determined with a Z2 Coulter Counter and Size Analyzer (Beckman Coulter, Inc., Palo Alto, Calif., U.S.A.). Total RNA for RT-PCR was isolated from 2 million cells from each of the three zones cells using the RNeasy Mini Kit (Qiagen, Valencia, Calif., U.S.A.).

After isolation, chondrocytes from the three zones were plated onto separate 10 cm tissue culture dishes at a density of 10,000 cells/cm². Cells were incubated at 37° C. and 5% CO₂ in DMEM containing 10% fetal bovine serum, 0.4 mM proline, 50 μg/ml ascorbic acid, 10 mM HEPES, 0.1 mM non-essential amino acid, and 100 U/ml penicillin and 100 μg/ml streptomycin. Culture medium was changed twice weekly. When the cells reached 80-90% confluence, total RNA was extracted from cells in a single 10 cm culture dish.

Assessment of cell number and size was performed in three experiments at different times (n=3 per each layer from 3 animals). Cell number and size were counted using a Z2 Coulter Counter and cell viability was determined by Trypan Blue dye exclusion method. The greatest number of cells per gram of tissue was obtained from the upper zone [42.7 (±1.45)×10⁶ cells/gram], followed by the middle zone [24.2 (±2.57)×10⁶ cells/gram], and the lower zone [13.2 (±1.16)×10⁶ cells/gram] (U vs. M, p=0.000; U vs. L, p=0.000; and M vs. L, p=0.001). Cell sizes of the lower chondrocytes were the largest (diameter: 13.2±0.52 μm) followed by the middle chondrocytes (12.0±0.15 μm) and the upper chondrocytes (10.7±0.14 μm) (U vs. M, p=0.005; U vs. L, p=0.000; and M vs. L, p=0.01). These quantitative measurements were consistent with histologic observations of chondrocytes in native articular cartilage. The cell viabilities of chondrocytes from all three zones were greater than 97% and there was no difference among the three zones (p>0.05).

Growth kinetics for the three chondrocyte zones were also determined. Chondrocytes from each zone were plated at a density of 2500 cells/cm² in 12-well culture plates. Cells were cultured for twelve days at 37° C. and 5% CO₂, and medium was changed twice a week. At a specific time each day, cells from three wells were trypsinized and counted using a Z2 Coulter Particle Count and Size Analyzer. The number and size of cells were calculated as a mean and standard deviation (n=9). Population doubling and population doubling time were determined using the following equation: PD=3.32
[log(cell#_{harvested})−log(cell#_{plated})].

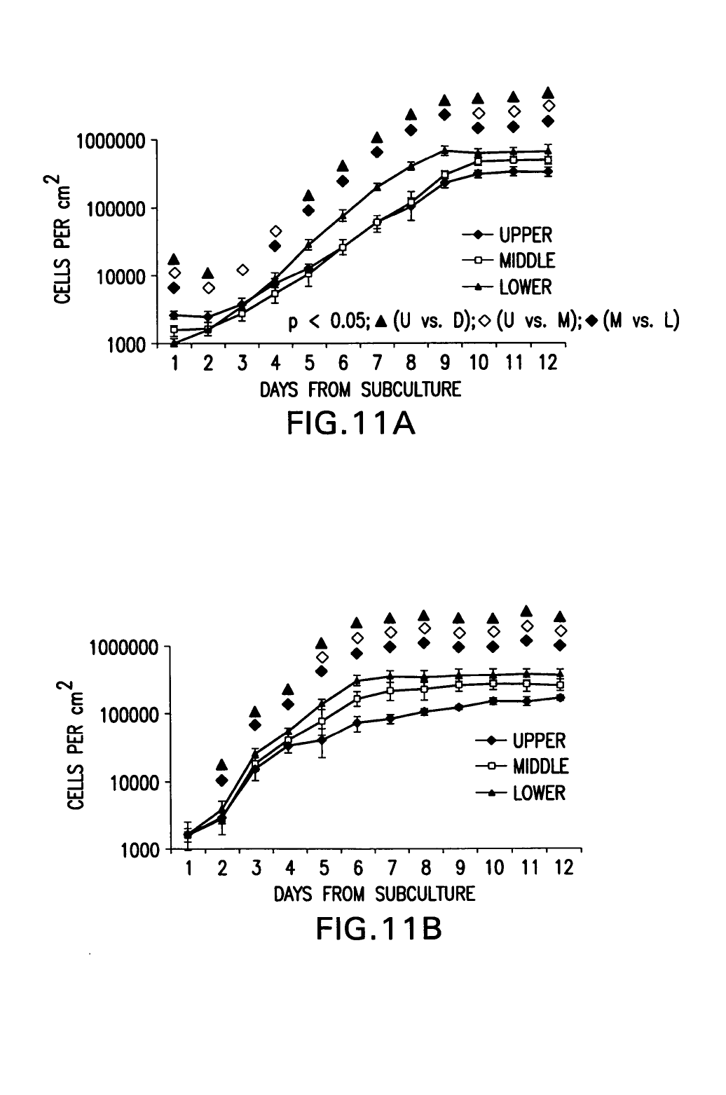

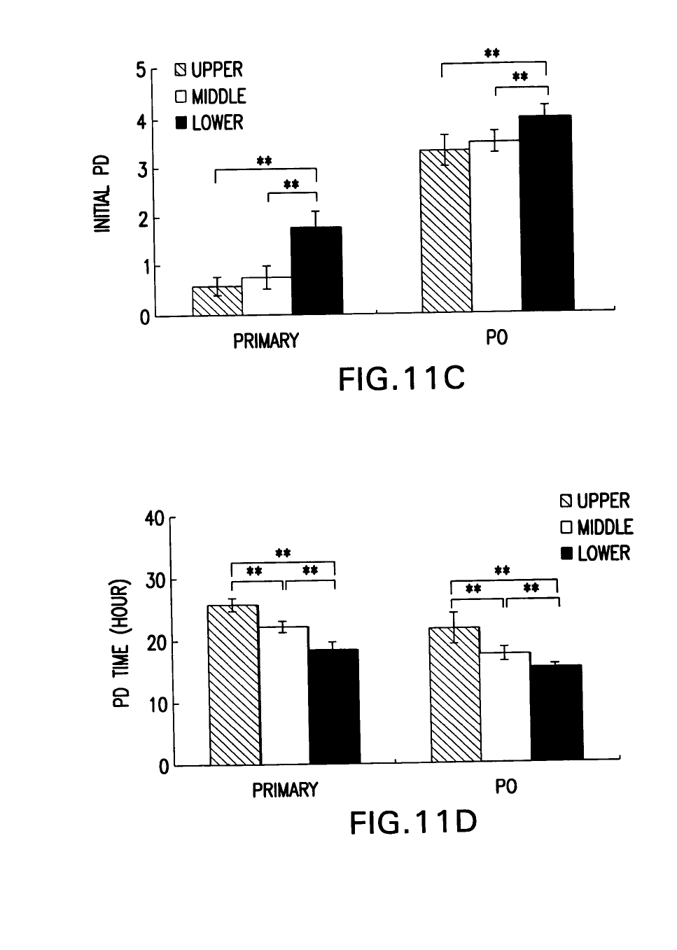

When the primary isolated cells (P0) from each zone were cultured in monolayer, they demonstrated significant differences in growth kinetics as shown in FIG. 11. The cells of the lower zone had the greatest proliferative capacity, as suggested by evaluation of the lag phase, population doubling time, and saturation density. The lower cells did not exhibit a lag phase of growth as the upper and middle cell populations (FIGS. 11A, 11C). The number of population doublings of the primary cells in the first three days of culture was the greatest in the lower cells (1.8), followed by the middle (0.8) and the upper (0.6) cells. There was no lag phase in the plated cells. During the exponential growth phase, the lower chondrocytes demonstrated a faster population doubling time (18.8±1.1 hours) than the middle (22.4±0.9 hours) and upper chondrocytes (26.1±1.1) (p=0.000 in all three comparisons: U vs. M; U vs. L; and M vs. L) (FIG. 11D). The differences in population doubling time among the three layers were maintained in the plated cells.

Genotypic Characterization of Harvested Chondrocytes

The RT-PCR for the three cell populations was also obtained. One microgram of total RNA per 20 μl reaction was reverse transcribed into cDNA using the SuperScript First-Strand Synthesis System (Invitrogen, Grand Island, N.Y., U.S.A.). One microliter of cDNA sample was subsequently amplified at an annealing temperature of 55° C. for 35 cycles using the Takara Ex Taq DNA polymerase premix (Takara Bio Inc, Japan). Cartilage specific primers included type II collagen (F-gtggagcagcaagagcaagga, R-cttgccccacttaccagtgtg), aggrecan (F-gccttgagcagttcaccttc, R-ctcttctacggggacagcag), COMP (F-caggacgactttgatgcaga, R-aagctggagctgtcctggta), and type IX collagen (F-gtgttgctggtgaaaagggt, R-gggatcccactggtcctaattc). Two house-keeping genes, β-actin (F-tggcaccacaccttctacaatgagc, R-gcacagcttctccttaatgtcacgc) and GAPDH (F-gcctggtcaccagggctgc, R-tgctaagcagttggtggtgca) were used as an internal control. PCR products were separated by electrophoresis at 100 V on a 2% agarose gel in TAE buffer.

The gene expression of the cartilage specific markers differed among the cells from different zones and the pattern of the changes with plating was also different as shown in FIG. 12. Type II collagen expression of the upper chondrocytes was notably lower than the middle and lower chondrocytes. The aggrecan expression of primarily isolated cells had no remarkable differences among the zones and slight decreases were observed upon plating. In the primarily isolated cells, the expression level of type IX collagen of the lower cells was the strongest, followed by the middle and upper cells. This trend was maintained even upon plating. The gene expression of COMP was higher in the primarily isolated lower cells than in the upper and middle cells.

Evaluation of Non-Layered Tissue Constructs

To compare the matrix synthesis in 3-dimensional culture, chondrocytes from different zones were encapsulated separately in photopolymerizing gels. These three tissue constructs were similar to the prior art non-layered tissue construct shown in FIG. 9, except that the tissue construct of FIG. 9 contained chondrocytes 11, 12 and 13 from each zone in one hydrogel. In the present case, each of the non-layered tissue constructs in accordance with this example contained chondrocytes from either the superficial STZ zone, the middle zone, or the deep zone.

The hydrogel solution used in this example was prepared by mixing 10% weight/volume (w/v) of poly(ethylene glycol) diacrylate (PEGDA, Shearwater Corp., Huntsville, Ala.) in sterile PBS with 100 U/ml of penicillin and 100 μg/ml streptomycin (Gibco, Invitrogen Corporation, Carlsbad, Calif.). The photoinitiator, Igracure 2959 (Ciba Specialty Chemicals Corporation, Tarrytown, N.Y.) was added to the PEGDA solution and mixed thoroughly to make a final concentration of 0.05% w/v. Immediately prior to photoencapsulation, chondrocytes were resuspended in the solution to make a concentration of 20×10⁶ cells/ml and were gently mixed to make a homogeneous suspension. One hundred microliters of cell/polymer/photoinitiator suspension were transferred into cylindrical molds with a 6 mm internal diameter and exposed for 5 minutes to long-wave, 365 nm UV light at 4 mW/cm² (Glowmark Systems, Upper Saddle River, N.J.). The mono-layered hydrogels were then removed from their molds, and incubated in separate wells of 12-well plates. Culture medium was changed twice a week. After 3-week culture, wet weights (ww) and dry weights (dw) after 48 hours of lyophilization were obtained from constructs from each zone (n=9). The dried constructs were crushed with a tissue grinder (Pellet Pestle Mixer; Kimble/Kontes) and digested in 1 ml of papain solution (Worthington Biochemical Corporation). DNA, GAG, collagen assays were performed in the same methods described above. Results of GAG and collagen assays were normalized to DNA content.

Biochemical assays of single-layered PEGDA hydrogels revealed that the chondrocytes from each zone differed in matrix synthesis even after 3-dimensional culture (n=3). GAG synthesis by the middle and lower chondrocytes was significantly greater than that of the upper chondrocytes, by 26% and 46% respectively. In addition, the lower chondrocytes synthesized 55% and 35% more collagen than the upper and middle chondrocytes, respectively.

The Making and Evaluation of a Three-layered Tissue Construct

The steps to create multi-layered tissue constructs are illustrated in FIG. 7. First, the hydrogel solution used to make the mono-layered tissue constructs described above was used to make the photopolymer-cell suspensions A, B and C. As also discussed above, the hydrogel solution included photoinitiator, Igracure 2959, at a concentration of 0.05% w/v. Briefly, 120 μl of the photopolymer-cell suspension A containing lower chondrocytes (20×10⁶ cells/ml) was placed in a 8 mm cylindrical mold and allowed to polymerize under the UVA lamp for 3 minutes (such that the solution only partially gelled), then 120 μl of photopolymer-cell suspension with middle chondrocytes (20×10⁶ cells/ml) was added and exposed to WVA light for 3 minutes. Finally, 120 μl of photopolymer-cell suspension C containing upper chondrocytes (20×10⁶ cells/ml) was added and exposed to UVA light for 3 minutes. To ensure that all three layers were completely gelled, the three layers were subsequently exposed to the UVA light for an additional minute. The resulting multi-layered composite gels, also referred to as tissue constructs, were removed from the mold and incubated in separate 12 well plates.

To confirm that the encapsulated cells stayed in the respective layer, cell tracking protocols (CellTracker™ Probes, Molecular Probes, Eugene, Oreg., U.S.A.) were performed 3 days after encapsulation according to the manufacturer's protocols. Briefly, the upper and lower chondrocytes were labeled by incubating for 30 minutes in 10 ml DMEM media with 5 μM CellTracker Green CMFDA. CellTracker Orange CMTMR was used for labeling of the middle chondrocytes in the same way. Labeled cells were encapsulated to make multilayered constructs in the same way described above. Constructs were harvested for fluorescence microscopy immediately and 3 days after encapsulation. Fluorescence microscopy was performed using a fluorescein optical filtuer (485±10 nm) for CMFDA and a rhodamine optical filter (530±12.5 nm) for CMTMR.

Cell tracking studies on the encapsulation day and 3 days after encapsulation confirmed that the encapsulated cells had stayed in the respective layer. A small amount of cell settling was observed in the lower sections of the gels but there was no cell migration between the layers of the constructs from day 0 to day 3.

Cell viability of the encapsulated cells was evaluated with Live/Dead Viability/Cytotoxicity Kit (Molecular Probes, Eugene, Oreg., U.S.A.). Briefly, thin slices (100-200 μm) of three layers were prepared with a surgical blade from the constructs after 3 and 21 day culture. The slices were incubated for 30 minutes in Live/Dead assay reagents (2 μM calcein AM and 4 μM. Fluorescence microscopy was performed using a fluorescein optical filtuer (485±10 nm) for calcein AM and a rhodamine optical filter (530±12.5 nm) for Ethidium homodimer-1.

Cell viability assay of multi-layered hydrogel constructs revealed that cells survived photoencapsulation and remained viable in tri-layered constructs that were approximately 8 mm thick. No differences among the cells from different layers were found in cell viability after 3 and 21 day culture.

After 3 week culture, the three-layered tissue constructs were harvested for histologic and immunohistochemical studies. The hydrogels were fixed overnight in 2% paraformaldehyde at 4° C. and transferred to 70% ethanol until embedded in paraffin according to standard histological technique. Sections were stained with Safranin-O/Fast Green. Immunohistochemistry was performed using the Histostain-SP kit (Zymed Laboratories Inc., San Francisco, Calif., U.S.A.) following the manufacturer's protocol. Rabbit polyclonal antibody to type II collagen (Research Diagnostics Inc., Flanders, N.J., U.S.A.) was used as the primary antibody.

Safranin-O staining revealed that each layer of multi-layered constructs showed similar histologic findings to the relevant zone of native cartilage. The upper layer had small cells with a flattened or ellipsoidal cellular morphology whereas middle and lower layers had large cells with an oval or round cellular morphology. The diameter of pericellular matrix stained with Safranin-O was greatest in the lower layer, followed by the middle and upper layers.

Immunohistochemisty for type II collagen showed that the location of collagen deposition was similar to that of proteoglycan synthesis shown in Safranin-O staining. The diameter of positive staining pericellular areas was the greatest in the lower layer. Many cells in the upper layer had no positive staining in the pericellular regions.

Thus, the above results show that viable multi-layered tissue constructs can be engineered in accordance with the present invention so as to mimic physiological multi-layered tissue architecture. The above results show that encapsulated cells do not migrate, but they do retain the multi-layered architecture over time. Furthermore, the encapsulated cells in each layer appear to function as if they remained in the respective tissue zone from which they were originally harvested.

### ILLUSTRATIVE EXAMPLE 2

### Multi-Layered Tissue Construct Encapsulating Chondrocytes from Two Zones of Articular Cartilage

In this illustrative example, a two-layered tissue construct, such as shown in FIG. 1, is created using a photopolymer, a photoinitiator and WVA radiation to effect crosslinking and hydrogel formation, and the encapsulated cells are chondrocytes harvested from two different tissue zones (i.e., superficial and deep) in mammalian articular cartilage. The present two-layered tissue construct, while formed in the cavity of a mold, is suitable for transplantation and could have been engineered in situ directly in the cavity of an articular joint defect.

First, chondrocytes corresponding to the two different cell types (i.e., superficial STZ zone chondrocytes and the deep zone chondrocytes) to be encapsulated in the different hydrogel layers were separately harvested using the methods described in the first illustrative example. Next, a hydrogel solution using PEGDA and the photoinitiator Igracure 2959, in accordance with the procedure described for the first illustrative example, is prepared. Next, superficial chondrocytes and deep chondrocytes are added separately to an amount of the hydrogel solution to make two different photopolymer-cell suspensions.

Next, 120 μl of the photopolymer-cell suspension containing deep chondrocytes (20×10⁶ cells/ml) was placed in a 8 mm cylindrical mold and allowed to polymerize under the UVA lamp for 3 minutes (such that the solution only partially gelled), then 120 μl of photopolymer-cell suspension with superficial chondrocytes (20×10⁶ cells/ml) was added and exposed to UVA light for 3 minutes. To ensure that all three layers were completely gelled, the three layers were subsequently exposed to the UVA light for an additional minute. The resulting two-layered tissue constructs were removed from the mold and incubated in separate well plates for six weeks in a complete medium.

After six weeks incubation, the shear strength and the peel strength of the two-layered tissue constructs, created in accordance with illustrative example two of the present invention, were tested and compared to various non-layered (i.e., mono-layered) tissue constructs. In this way, a comparison of the mechanical characteristics of a multi-layered tissue construct was made to the mechanical strength characteristics of various mono-layered tissue constructs

Specifically, the mono-layered tissue constructs were each made using the same hydrogel solution using the same photopolymer and photoinitiator as was used to make each layer of the two-layered tissue construct. However, four different mono-layered tissue constructs were made by adding cells to the hydrogel solution so that a photopolymer-cell suspension containing 20×10⁶ cells/ml was prepared for each cell type, then 120 μl of each photopolymer-cell suspension containing chondrocytes at a concentration of 20×10⁶ cells/ml was placed in a 8 mm cylindrical mold and allowed to polymerize under the UVA lamp for 2 minutes (such that the solution only partially gelled), followed by polymerization under the UVA lamp for an additional three minutes to ensure complete polymerization. Each mono-layered was then removed from the mold and incubated for six weeks in a complete medium.

The composition of the cells in the four different types of mono-layered tissue constructs were as follows: S: superficial chondrocytes only; D: deep chondrocytes only; A: all chondrocytes (i.e., superficial, middle and deep zone chondrocytes such as shown in FIG. 9), and S-D mixed: equal numbers of superficial and deep zone chondrocytes.

The mechanical tests for shear strength and for compressive strength were performed using the RFS3 Mechanical Tester (TA Instruments Inc.). Strain sweeping was first performed to determine the linear visco-elastic zone (i.e., strain range) for each chondrocyte-hydrogel tissue construct. The equilibrium shear modulus and Young's modulus for each construct was determine from the following two tests, respectively: 1) shear stress relaxation with a magnitude of 1% in a step mode, and 2) axial compressive test of 10% strain in 400 sec.

The results of the mechanical testing described above are tabulated in Table 1 below. S/D (whole) corresponds to the two-layered tissue construct made in accordance with the present illustrative example, whereas all of the remaining constructs tested are mono-layer constructs. In Table 1, n equals the number of constructs tested and shear strength and peel strength are measured in kPa.

TABLE 1

| Tissue Construct | n | Shear Modulus (kPa) | Young's Modulus (kPa) |
|---|---|---|---|
| S/D (whole) | 2 | 10.1 ± 0.4 | 35.9 ± 3.3 |
| S (alone) | 2 | 4.9 ± 0.5 | 25.4 ± 5.2 |
| D (alone) | 3 | 5.1 ± 1.0 | 22.7 ± 11.3 |
| A (See FIG. 9) | 2 | 3.3 ± 0.1 | 16.0 ± 3.1 |
| S − D (mixed) | 1 | 3.7 | 20.6 |

As shown from the data in Table 1, the measured shear modulus and Young's modulus for the two-layered tissue construct (S/D) was significantly greater than for any of the mono-layered tissue construct, including the prior art mono-layered tissue construct corresponding to FIG. 9. In other words, the two-layered tissue construct was stronger and had greater shear and peel strength characteristics than the mono-layered tissue constructs. This illustrative example proves the mechanical advantage of making tissue implants that closely mimic the actual physiologic architecture of a layered tissue, such as articular cartilage, over mono-layered implant structures that poorly resemble layered tissue structures.

### ILLUSTRATIVE EXAMPLE 3

### Multi-Layered Tissue Construct Encapsulating Chondrocytes from Two Zones of Articular Cartilage

In this example, a two-layered tissue construct is formed in situ directly on a cartilage tissue defect in a human patient. Superficial and deep zone chondrocytes are harvested and cultured in advance from either the patient (i.e., autologous donor) or from a cadaver by using the harvesting technique for chondrocytes described above. Next, hydrogel solution is prepared by thoroughly mixing 10% w/v of either PEODA or PEGDA and the photoinitiator Igracure 2959 (final concentration 5% w/v) in sterile PBS. Antibiotics and a growth factor are also included in the hydrogel solution. Specifically, 100 U/ml of penicillin and 100 μg/ml of streptomycin and transforming growth factor (TGF-β, RDI, 150 ng/ml) are added to the hydrogel solution. Next, the superficial and deep chondrocytes are separately resuspended in the hydrogel solution at a concentration of 20 million cells/ml, and gently mixed, so there is a homogenous hydrogel suspension containing superficial chondrocytes and a separate homogenous hydrogel suspension containing deep chondrocytes.

Using a standard orthopedic surgical protocol known to surgeons in the art, the patient's knee joint is prepped and draped in the usual sterile fashion. Although the present method can be used to treat any surgically accessible joint, it is most useful for treating knee pathology. Using a suitable arthroscope, the surgeon accesses the joint space through a first incision and visualizes the articular defect to be treated. The defect is surgically debrided, if necessary, by the surgeon using a surgical tool inserted through a port in the arthroscope or by providing a surgical tool inserted through a second incision in the knee.

Next, the surgeon applies a volume of the hydrogel suspension containing the deep zone chondrocytes in sufficient quantity to fill the floor of the defect. This first hydrogel suspension is supplied either through a port in the arthroscope or through a tube temporarily inserted into the joint space through the second incision. The first hydrogel suspension is then partially gelled by exposure to long-wave, 365 nm UV light at 4 mW/cm² (Acticure) for 3-5 minutes to form a base hydrogel layer. The UV light is applied either through the fiber optics of the arthroscope or through a separate fiber optic device temporarily inserted through the second incision.

The surgeon then applies a volume of the hydrogel suspension containing the superficial zone chondrocytes on top of the partially gelled base hydrogel. The surgeon applies this second hydrogel suspension in sufficient quantity to fully cover the upper surface of the base hydrogel and to fill the cartilage defect. The second hydrogel suspension is supplied either through a port in the arthroscope or through a tube temporarily inserted into the joint space through the second incision. The second hydrogel suspension is then partially gelled by exposure to long-wave, 365 nm UV light at 4 mW/cm² (Acticure) for 3-5 minutes to form a top hydrogel layer. The UV light is applied either through the fiber optics of the arthroscope or through a separate fiber optic device temporarily inserted through the second incision.

Lastly, the surgeon may apply the UV light for an additional 1-5 minutes, if deemed necessary, to ensure complete gelling of both top and base hydrogel layers. The surgeon then removes all surgical instruments from the patient's knee and closes all incisions with suture and/or surgical staples. The patient is transferred to postoperative recovery where post-operative care protocols are continued.

While the present invention has been described generally, followed by a description of several illustrative examples, those skilled in the art would realize that these embodiments are not limiting. For example, the present invention could be used to make a four or five layered tissue construct wherein one of the hydrogel layers contains a cell type that is very different from the others, such as when a base layer is made to contain bone cells and the remaining hydrogel layers each contain a different type of chondrocyte cell.

In addition, while each layer of the multi-layered tissue constructs engineered in accordance with the present invention have been described as having either no cells or a single cell type encapsulated in the hydrogel of each layer, the present invention is not limited in this manner. It is within the scope of the present invention to make a multi-layered tissue construct that has at least one layer with two or more different cell types encapsulated in the hydrogel of that layer. It is also within the scope of the present invention to make a multi-layered tissue construct that has two or more layers wherein each layer encapsulates one or more different cell types.

While the present invention has been described with reference to certain preferred embodiments, one of ordinary skill in the art will recognize that additions, deletions, substitutions, modifications and improvements can be made while remaining within the spirit and scope of the present invention as defined by the appended claims.

1. A method of producing a multi-layered tissue construct comprising the steps of:
providing a first polymerizable mixture, including, optionally, a first polymerization initiator;
providing a second polymerizable mixture, including, optionally, a second polymerization initiator;
wherein one of the first and second mixtures comprises a cells;
placing a volume of the first mixture in a space, then crosslinking the first mixture for a first predetermined time until the first mixture forms an at least partially gelled first layer; and
placing a volume of the second mixture in the space with the at least partially gelled first layer, then crosslinking the second mixture for a second predetermined time until the second mixture is at least partially gelled to form a second layer.

2. (Canceled)

3. A method according to claim 1, further comprising the step of adding a suspension of cells to a surface of the at least partially gelled first layer, before the step of placing the volume of the second mixture in the space.

4. A method according to claim 1, wherein one of the first mixture and the second mixture further comprises a bioactive substance selected from the group consisting of: a nutrient, a cellular mediator, a growth factor, a compound which induces cellular differentiation, a bioactive polymer, a gene vector, or a pharmaceutical.

5. A method as recited in claim 1, wherein the step of providing the first mixture includes mixing the first polymerizable mixture with first cells to form a first polymer-cell suspension, and the step of providing the second mixture includes mixing the second polymerizable mixture with second cells to form a second polymer-cell suspension.

6. A method as recited in claim 5, further comprising the step of:
additionally crosslinking the first mixture and the second mixture until the first layer and the second layer further polymerize to form an integrated multi-layered gel.

7. A method as recited in claim 5, wherein the first cells and the second cells are selected from the group of cell types consisting of superficial zone chondrocytes, middle zone chondrocytes, and deep zone chondrocytes.

8. A method as recited in claim 7, wherein the first cells are a cell type different from the second cells.

9. A method as recited in claim 8, wherein the first cells are deep zone chondrocytes and the second cells are superficial zone chondrocytes.

10. A method as recited in claim 7, further comprising the step of:
harvesting mammalian articular cartilage and excising tissue specimens corresponding to an upper zone, a middle zone and a deep zone of the cartilage; and
separately digesting the tissue specimens from the upper zone, the middle zone and the deep zone respectively to isolate upper zone chondrocytes, middle zone chondrocytes and deep zone chondrocytes.

11. A method as recited in claim 5, wherein the cell concentration of each suspension is approximately 20 million cells/cc.

12. A method as recited in claim 6, further comprising the step of:
incubating the multi-layered gel in a complete media for a predetermined incubation period to form the multi-layered tissue construct.

13. A method as recited in claim 5, further comprising the steps of:
providing a third polymerizable mixture, including, optionally, a third polymerization initiator, wherein the third polymerizable mixture is mixed with third cells to prepare a third polymer-cell suspension; and
placing a volume of the third mixture in the space with the at least partially gelled first layer and the at least partially gelled second layer, then crosslinking the third mixture for a third predetermined time until the third mixture is at least partially gelled to form a third layer.

14. A method as recited in claim 13, further comprising the step of:
additionally crosslinking the first layer, the second layer and the third layer to form an integrated multi-layered gel.

15. A method as recited in claim 14, wherein the first cells, the second cells and the third cells are selected from the group of cell types consisting of superficial zone chondrocytes, middle zone chondrocytes, and deep zone chondrocytes.

16. A method as recited in claim 15, wherein the first cells, the second cells and the third cells are selected to be different cell types.

17. A method as recited in claim 15, wherein the first cells are deep zone chondrocytes, the second cells are middle zone chondrocytes, and the third cells are superficial zone chondrocytes.

18. A method as recited in claim 17, further comprising the step of:
incubating the multi-layered gel in a complete media for a predetermined incubation period to form the multi-layered tissue construct.

19. A method as recited in claim 13, further comprising the steps of:
additionally crosslinking the first layer, the second layer and the third layer until the first layer, the second layer and the third layer completely polymerize to form a multi-layered gel; and
optionally incubating the multi-layered gel in a complete media for a predetermined period of time to form the multi-layered tissue construct.

20. A method as recited in claim 5, wherein the first polymerizable mixture and the second polymerizable mixture both include photopolymerizable poly(ethylene glycol) diacrylate dissolved in solvent, which is phosphate buffered saline, to make a 10% w/v solution, and the first polymerization initiator is added to the first mixture and the second polymerization initiator is added to the second mixture, wherein both the first polymerization initiator and the second polymerization initiator are the same photoinitiator, and each suspension has a concentration of 20 million cells/cc.

21. A method as recited in claim 20, wherein the photoinitiator is Igracure 2959 mixed to a concentration of 0.05% w/v in each suspension.

22. A method as recited in claim 21, wherein crosslinking of the first polymerizable mixture is controlled by exposure to external radiation and crosslinking of the second polymerizable mixture is controlled by exposure to the external radiation.

23. (Canceled)

24. A multi-layered tissue construct comprising:
a first layer comprising a first hydrogel; and
a second layer comprising a second hydrogel, wherein the first layer is connected to the second layer at a first transition zone and wherein at least one of the first layer and the second layer further comprises cells.

25. A multi-layered tissue construct as recited in claim 24, wherein the first layer comprises cells of a first cellular type encapsulated in the first hydrogel.

26. A multi-layered tissue construct as recited in claim 25, wherein the second layer comprises cells of a second cellular type encapsulated in the second hydrogel, and the first cell type is different from the second cell type.

27. A multi-layered tissue construct as recited in claim 26, further comprising:
a third layer comprising cells of a third cellular type encapsulated in a third hydrogel, wherein a second transition zone connects the third layer to the second layer, and the third cell type is different from the second cell type.

28. A multi-layered tissue construct as recited in claim 27, wherein the first cell type is a deep zone chondrocyte, the second cell type is a middle zone chondrocyte, and the third cell type is a superficial zone chondrocyte.

29. A multi-layered tissue construct as recited in claim 28, wherein the first hydrogel, the second hydrogel and the third hydrogel include photopolymerized poly(ethylene glycol) diacrylate.

30. A multi-layered tissue construct as recited in claim 26, wherein the first cell type is a deep zone chondrocyte and the second cell type is a superficial zone chondrocyte.

31. A multi-layered tissue construct as recited in claim 30, wherein the first hydrogel and the second hydrogel both include photopolymerized poly(ethylene glycol) diacrylate.

32. (Canceled)

33. (Canceled)

34. (Canceled)

35. A multi-layer tissue construct as recited in claim 25, wherein the first layer further comprises cells of a second cellular type encapsulated in the first hydrogel.

36. A multi-layered tissue construct as recited in claim 24, wherein the first layer comprises a bioactive substance selected from the group consisting of: a nutrient, a cellular mediator, a growth factor, a compound which induces cellular differentiation, a bioactive polymer, a gene vector, or a pharmaceutical.

37. A multi-layered tissue construct as recited in claim 25, wherein the first layer also includes a bioactive substance.

38. A multi-layered tissue construct as recited in claim 25, wherein the second layer includes a bioactive substance.

39. (Canceled)

40. (Canceled)

41. (Canceled)

42. (Canceled)

43. (Canceled)

44. (Canceled)

45. (Canceled)

46. (Canceled)

47. (Canceled)

48. (Canceled)

49. (Canceled)

50. (Canceled)

51. (Canceled)

52. (Canceled)
